SEQUENCE LISTING (22 sequences)

SEQ ID NO: 1 - DNA, 76 bp, Artificial Sequence
  GFP construct
cccaccctcgtgaccaccctgacctacggcgtgcagtgcttcagccgctaccccgaccacatgaagcagcacgact

SEQ ID NO: 2 - protein, 25 aa, Artificial Sequence
  GFP construct
PTLVTTLTYGVQCFSRYPDHNKQHD

SEQ ID NO: 3 - DNA, 76 bp, Artificial Sequence
  BFP construct
cccaccctcgtgaccaccctgagccacggcgtgcagtgcttcagccgctaccccgaccacatgaagcagcacgact

SEQ ID NO: 4 - protein, 25 aa, Artificial Sequence
  BFP construct
PTLVTTLSHGVQCFSRYPDHNKQHD

SEQ ID NO: 5 - DNA, 71 bp, Artificial Sequence
  GFP construct
  CDS (1)..(69)
ctgcccgtgccctggcccaccctcgtgaccaccctgacctacggcgtgcagtgcttcagccgctaccccga

SEQ ID NO: 6 - protein, 24 aa, Artificial Sequence
  BFP construct
LPVPWPTLVTTLSHGVQCFSRYPD

SEQ ID NO: 7 - DNA, 66 bp, Artificial Sequence
  GFP construct
  CDS (1)..(66)
gtgccctggcccaccctcgtgaccaccctgacctacggcgtgcagtgcttcagccgctaccccgac

SEQ ID NO: 8 - protein, 22 aa, Artificial Sequence
  BFP construct
VPWPTLVTTLSHGVQCFSRYPD

SEQ ID NO: 9 - protein, 22 aa, Artificial Sequence
  GFP construct
VPWPTLVTTLTYGVQCFSRYPD

SEQ ID NO: 10 - protein, 22 aa, Artificial Sequence
  BFP construct
VPWPTLVTTLSHGVQCFSRYPD

SEQ ID NO: 11 - protein, 21 aa, Artificial Sequence
  GFP mutant
VPWPTLVTTLKHACSASAATP

SEQ ID NO: 12 - protein, 22 aa, Artificial Sequence
  GFP construct
VPWPTLVTTLTYGVQCFSRYPD

SEQ ID NO: 13 - protein, 22 aa, Artificial Sequence
  BFP construct
VPWPTLVTTLSHGVQCFSRYPD

SEQ ID NO: 14 - protein, 20 aa, Artificial Sequence
  GFP mutant
VPWPTLVTTLTACSASAATP

SEQ ID NO: 15 - protein, 22 aa, Artificial Sequence
  GFP construct
VPWPTLVTTLTYGVQCFSRYPD

SEQ ID NO: 16 - protein, 22 aa, Artificial Sequence
  BFP construct
VPWPTLVTTLSHGVQCFSRYPD

SEQ ID NO: 17 - protein, 21 aa, Artificial Sequence
  GFP mutant
VPWPTLVTTLTRRAVLQPLPR

SEQ ID NO: 18 - protein, 22 aa, Artificial Sequence
  GFP construct
VPWPTLVTTLTYGVQCFSRYPD

SEQ ID NO: 19 - protein, 22 aa, Artificial Sequence
  BFP construct
VPWPTLVTTLSHGVQCFSRYPD

SEQ ID NO: 20 - protein, 22 aa, Artificial Sequence
  GFP mutant
VPWPTLVTTLTSGHHRRLPYPN

SEQ ID NO: 21 - DNA, 21 bp, Artificial Sequence
  Primer
gtgctgtctcatcattttggc

SEQ ID NO: 22 - DNA, 20 bp, Artificial Sequence
  Primer
tcgtgctgcttcatgtggtc

(19) United States
(12) Patent Application Publication
(10) Pub. No.: US 2021/0363546 A1
(43) Pub. Date: Nov. 25, 2021

(54) IN VIVO HOMOLOGY DIRECTED REPAIR IN HEART, SKELETAL MUSCLE, AND MUSCLE STEM CELLS

(71) Applicant: President and Fellows of Harvard College, Cambridge, MA (US)

(72) Inventors: Amy J. Wagers, Cambridge, MA (US); Kexian Zhu, Medford, MA (US)

(73) Assignee: President and Fellows of Harvard College, Cambridge, MA (US)

(21) Appl. No.: 17/052,798

(22) PCT Filed: May 3, 2019
(86) PCT No.: PCT/US19/30748

§ 371 Date: Nov. 3, 2020

Related U.S. Application Data
(60) Provisional application No. 62/666,685, filed on May 3, 2018.

(51) Int. Cl. C12N 15/90; C12N 9/22

(52) U.S. Cl. CPC C12N 15/907; C12N 2510/00; C12N 2750/14142; C12N 9/22

(57) ABSTRACT

Disclosed are methods of genomic modification of skeletal and cardiac muscle using sequence-targeting nucleases and a donor sequence delivered via a virus.

### RELATED APPLICATIONS

This application claims the benefit of U.S. Provisional Application Ser. No. 62/666,685, filed May 3, 2018, the contents of which are hereby incorporated by reference in its entirety.

### BACKGROUND OF THE INVENTION

Sequence-targeting nuclease such as CRISPR/Cas9 provide powerful tools to edit mammalian genomes by engaging cellular mechanisms of DNA double strand break (DSB) repair. Non-homologous end joining (NHEJ) and homology-directed repair (HDR) are the major pathways used by cells to mend nuclease-generated DSBs and prevent genomic lesions and cell death. While NHEJ is active throughout the cell cycle, and in non-dividing cells, this error-prone pathway produces variable sequence outcomes due to highly unpredictable nucleotide insertions and deletions.

In contrast, HDR offers more precise gene-editing outcomes, as well as the unique capacity to introduce entirely new sequence elements, but HDR is generally believed to be inefficient in post-mitotic organs and requires homologous DNA present on either endogenous chromosomes or exogenous templates. While recent studies have investigated the use of CRISPR-induced HDR in cultured cells, zygotes and with local delivery to specific tissues, the feasibility of achieving multi-organ HDR in vivo in postnatal mammals has not been tested. In addition, whether in vivo HDR targeting could be achieved in regenerative stem cells, providing a reservoir of edited cells to support ongoing tissue turnover and repair, has yet to be explored.

### SUMMARY OF THE INVENTION

The inventors have surprisingly and unexpectedly found that postnatal cardiac muscle, skeletal muscle, and muscle stem cells undergo templated homology directed repair (HDR, also referred to as homologous recombination) at different developmental time points in mice. This provides an unexpected opportunity for precise, targeted gene replacement by HDR in skeletal and cardiac muscles, both largely post-mitotic tissues that have been widely considered to be inaccessible by this approach. To our knowledge, this data provides the first demonstration of significant in vivo HDR-editing in the postnatal heart via systemic AAV delivery of CRISPR/Cas9, and represents a substantial improvement over previously reported HDR editing rates achievable in skeletal muscle via local, intramuscular delivery. The invention described herein also provides the first demonstration of successful HDR-editing in tissue stem cells within their native niche, which will uniquely enable directed manipulation of stem cell genomes therapeutically and experimentally, without the need to isolate, expand or transplant these rare cells. Ultimately, the ability to inscribe irreversible and potentially enduring precise genome modification in the neonatal mammalian heart and postnatal mammalian skeletal muscle satellite cells opens exciting new avenues for future therapeutic interventions for many currently intractable cardiac and muscle diseases, including for Duchenne Muscular Dystrophy (DMD).

Some aspects of the invention are directed to a method of modifying the genome of a muscle precursor cell in vivo (e.g., in the muscle precursor niche) in a subject, comprising contacting the muscle cell with one or more viruses, wherein the one or more viruses transduce a nucleic acid sequence encoding a sequence-targeting nuclease in the muscle precursor cell and transduce a donor template in the muscle precursor cell, wherein the modification comprises the insertion of a nucleotide sequence corresponding to a nucleotide sequence of the donor template.

In some embodiments, the one or more viruses comprise a first virus which transduces a nucleic acid sequence encoding a sequence-targeting nuclease and a donor template. In some embodiments, the one or more viruses comprise a first virus which transduces a nucleic acid sequence encoding a sequence-targeting nuclease, and a second virus which transduces a donor template. In some embodiments, the one or more viruses comprise a first virus which transduces a nucleic acid sequence encoding a sequence-targeting nuclease, and a second virus which transduces a donor template and one or more gRNAs (e.g., one or two). In some embodiments, the sequence-targeting nuclease is a Zinc-Finger Nuclease (ZFN), a Transcription activator-like effector nuclease (TALEN), a Cas nuclease (e.g., Cas9 nuclease), or a functional fragment thereof.

In some embodiments, the nucleic acid sequence encoding a sequence-targeting nuclease is transduced with a muscle precursor cell specific promoter, a constitutive promoter, or a ubiquitous promoter. In some embodiments, the nucleic acid sequence encoding a donor template and, optionally, one or more gRNAs, is transduced with the U6 or H1 promoter. In some embodiments, the muscle precursor cell is a muscle stem cell.

In some embodiments, at least 1% of muscle precursor cells in the subject are modified to comprise an insertion of a nucleotide sequence corresponding to a nucleotide sequence of the donor template. In some embodiments, the modification is of one allele. In some embodiments, the modification is of both alleles. In some embodiments, the subject (e.g., human or mouse) is not an infant, or juvenile, or under 30 years of age.

In some embodiments, the virus is AAV serotype 6, 8, 9, 10 or Anc80. In some embodiments, the virus is administered systemically to the subject or the virus is administered by intramuscular injection.

Some aspects of the disclosure are directed to a myofibre comprising nuclei (e.g., myonuclei) having genomes modified by the methods disclosed herein.

Some aspects of the disclosure are directed towards a method of modifying the genome of a cardiac cell in vivo in a subject, comprising contacting the cardiac cell with one or more viruses, wherein the one or more viruses transduce a nucleic acid sequence encoding a sequence-targeting nuclease in the cardiac cell, and transduce a donor template in the cardiac cell, wherein the modification comprises the insertion of a nucleotide sequence corresponding to a nucleotide sequence of the donor template, and wherein the cardiac cell is a DNA synthesizing cardiac cell or a replicating cardiac cell.

In some embodiments, the cardiac cell is selected from the group consisting of a mammalian postmitotic cardiomyocyte, a mammalian postmitotic cardiomyocyte capable of DNA synthesis without division/proliferation, a human postmitotic cardiomyocyte, a human postmitotic cardiomyocyte capable of DNA synthesis without division/proliferation, a cardiomyocyte precursor cell, a proliferating mesenchymal cardiac cell, a proliferating endothelial cardiac cell, and a cardiac progenitor cell.

In some embodiments, the subject (e.g., human or mouse) is an infant, or juvenile, or under 30 years of age if human. In some embodiments, the one or more viruses comprise a first virus which transduces a nucleic acid sequence encoding a sequence-targeting nuclease and a donor template. In some embodiments, the one or more viruses comprise a first virus which transduces a nucleic acid sequence encoding a sequence-targeting nuclease, and a second virus which transduces a donor template. In some embodiments, the one or more viruses comprises a first virus which transduces a nucleic acid sequence encoding a sequence-targeting nuclease, and a second virus which transduces a donor template and one or more gRNAs. In some embodiments, the sequence-targeting nuclease is a Zinc-Finger Nuclease (ZFN), a Transcription activator-like effector nuclease (TALEN), a Cas nuclease (e.g., Cas9 nuclease), or a functional fragment thereof. In some embodiments, the nucleic acid sequence encoding a sequence-targeting nuclease is transduced with a cardiac specific promoter, a ubiquitous promoter or a non-specific promoter.

In some embodiments, the virus is AAV serotype 6, 8, 9, 10 or Anc80. In some embodiments, at least 1.6% of the cardiomyocytes in the subject are modified.

Some aspects of the disclosure are directed to a cardiac tissue comprising cardiac muscle cells modified by the methods disclosed herein.

Some aspects of the disclosure are directed to a method of targeting a specific striated muscle type for genomic modification in vivo in a subject via homology directed repair, comprising systemically administering with one or more viruses, wherein the one or more viruses transduce a nucleic acid sequence encoding a sequence-targeting nuclease in striated muscle cells and transduce a donor template in striated muscle cells, wherein the modification comprises the insertion of a nucleotide sequence corresponding to a nucleotide sequence of the donor template, and wherein, due to the age of the subject, genomic modification preferentially occurs to at least one type of striated muscle. In some embodiments, the genomes of muscle cells (e.g., progenitor muscle cells) are preferentially modified. In some embodiments, the genomes of cardiac cells (e.g., proliferating or DNA synthesizing cardiac cells) are preferentially modified.

The above discussed, and many other features and attendant advantages of the present inventions will become better understood by reference to the following detailed description of the invention.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1J illustrate a GFP/BFP colour switch reporter system that enables discrimination and tracking of NHEJ- and HDR-edited myoblasts. (FIG. 1A) is a schematic of blue/green colour switch reporter for discriminating HDR vs. imprecise NHEJ. Imprecise NHEJ disrupts GFP fluorescence while HDR substitutions enable spectral shift from GFP to BFP and create a BtgI restriction site for RFLP analysis. (FIG. 1B) shows AAV constructs used for transfection and virus production. ITR, inverted terminal repeat; U6, U6 promoter; CMV, CMV promoter; NLS, nuclear localization signal; pA: polyA. (FIG. 1C) provides an experimental design. Skeletal muscle stem cells (satellite cells) were isolated from mice carrying a single CAG-GFP allele and transfected with plasmid constructs shown in (FIG. 1B). Transfected cells were expanded in culture, then sorted based on blue or green fluorescence for intramuscular transplantation into pre-injured recipient mice. (FIGS. 1D, 1E) are representative flow cytometric analysis of myoblasts transfected with gRNA-BFPtemplate alone (FIG. 1D, control) or myoblasts transfected with SaCas9 and gRNA-BFP template (FIG. 1E, experimental). (FIGS. 1F, 1G) show frequency (%) of CRISPR-HDR edited BFP+myoblasts (FIG. 1F) and CRISPR-NHEJ edited GFP-/BFP-myoblasts (FIG. 1G) in control or experimental cultures. Individual data points are shown overlaid with mean±SD and represent N=3 independent transfections. **p<0.01,***p<0.001, unpaired two-tailed t test, DF=4. (FIG. 1H) shows edited BFP+ SMPs retain myogenic potential. GFP+and BFP+ skeletal muscle progenitors were isolated by FACS and the Tibialis anterior (TA) muscles of mdx mice were injected with GFP+(bottom row) or CRISPR/Cas9-HDR-edited BFP+ (top row) stem cells. The TA was then examined by fluorescence detection of BFP or GFP. Scale bar, 50 um. Green, GFP; Blue, BFP; Red, Wheat Germ Agglutinin (WGA); White, TO-PRO-3. (FIG. 1I) shows PCR amplification at GFP locus followed by BtgI digestion of FACS sorted transfected cells. Three distinct populations found: GFP+SMPs (No editing), BFP+SMPs (HDR), and GFP-/BFP-SMPs (NHEJ). (FIG. 1J) shows sorted CRISPR/Cas9-HDR edited BFP+SMPs retain BFP expression following expansion. BFP+SMPs analyzed following two weeks of expansion.

FIGS. 2A-2G. illustrate systemic AAV-CRISPR enables in vivo CRISPR-NHEJ and CRISPR-HDR in the liver, heart and skeletal muscle of three week old GFP ^{+/-}mdx mice. (FIG. 2A) shows the experimental design. Mdx mice carrying a single CAG-GFP allele were injected with AAVs carrying GFPgRNA-BFP template only (control) or AAV-GFPgRNA-BFPtemplate plus AAV-SaCas9 (Dual CRISPR/Cas9 system). Organs were harvested 4 weeks later for fluorescence and genomic analyses. (FIGS. 2B, 2D, 2F) show representative fluorescence images for detection of CRISPR-NHEJ edited (GFP−/BFP−) and CRISPR-HDR edited (BFP+) cells in liver (FIG. 2B), heart (FIG. 2D), and tibialis anterior (skeletal muscle, FIG. 2F) after systemic co-injection of AAV-GFPgRNA-BFP template and AAV-SaCas9. Scale bars, 50 um. Green, GFP; Blue, BFP; Red, Wheat Germ Agglutinin (WGA); White, TO-PRO-3. (FIGS. 2C, 2E, 2G) shows frequency (%) of BFP+ (HDR-edited, left plots) or GFP−/BEP− (NHEJ-edited, right plots) cells in liver (FIG. 2C), heart (FIG. 2E) or tibialis anterior (FIG. 2G). NHEJ-editing could not be quantified for skeletal myofibres (i.e., myofibers) due to the high degree of multinucleation in this tissue, which prevents detection of green fluorescence loss unless nearly all myonuclei are targeted. N=4 mice for AAV-gRNA-temp and AAV-SaCas9 co-injection (experimental AAV-HDR group), N=3 for AAV-gRNA-temp injection alone (AAV-control group). 3 fields per tissue per mouse were quantified to generate the frequency data.

FIGS. 3A-3D) show satellite cells can be targeted in vivo via CRISPR-HDR and retain capacity to fuse and form myotubes in vitro. (FIG. 3A) shows representative flow cytometric analysis of skeletal muscle satellite cells from juvenile mdx mice injected intravenously with vehicle or AAV-GFPgRNA-BFPtemplate alone as controls or with AAV-GFPgRNA-BFPtemplate and AAV-SaCas9 to enable CRISPR-NHEJ and CRISPR-HDR. (FIGS. 3B, 3C) show frequency (%) of CRISPR-HDR edited BFP+ satellite cells (FIG. 3B) and CRISPR-NHEJ edited GFP−/BFP− satellite cells (FIG. 3C). Individual data points are shown overlaid with mean±SD; AAV-Cas9 and AAV-gRNA-temp (experimental), N=4 mice injected, AAV-gRNA-temp only (control), N=3 mice injected, Vehicle, N=3 mice injected. *p<0.05, not significant p=0.999 in (FIG. 39), p=0.7737 in (FIG. 3C), one-way ANOVA with Tukey's multiple comparisons test, DF=7. (FIG. 3D) shows representative fluorescence detection of myotubes differentiated from FACS sorted in vivo AAV-HDR injected GFP+ (unedited), BFP+ (HDR) and GFP−/BFP− (NHEJ) satellite cells. Scale bar, 100 um. Green, GFP; Blue, BFP; Red, myosin heavy chain (MHC); White, TO-PRO-3,

FIGS. 4A-4F shows delivery of color conversion system via AAV8 in P3 mice reveals tissue-dependent times restrictions on in vivo CRISPR-HDR targeting. (FIG. 4A) shows experimental design. P3 pups (wild-type and MDX) carrying a single CAG-GFP allele were injected with AAVs carrying GFPgRNA-BFP template only (control) or AAV-GFPgRNA-BFP template plus AAV-SaCas9. Organs were harvested 4 weeks later for fluorescence and genomic analyses. (FIGS. 4B, 4D, 4F) show representative fluorescence images for detection of CRISPR-NHEJ edited (GFP−/BFP−) and CRISPR-HDR edited (BFP+) cells in liver (FIG. 4B), heart (FIG. 4D), and tibialis anterior muscle (FIG. 4F) of GFP^{+/-}; mdx mice after intraperitoneal injection of AAV-GFPgRNA-BFP template and AAV-SaCas9 (experimental) or AAV-GFPgRNA-BFP template alone (control). Scale bars, 50 um. Green, GFP; Blue, BFP; Red, Wheat Germ Agglutinin (WGA); White, TO-PRO-3. (FIGS. 4C, 4E) show frequency (%) of GFP−/RFP− (NHEJ) and BFP+ (HDR) cells in liver (FIG. 4C) and heart (FIG. 4E) of treated GFP; mdx and wild-type (CAG-GFP) mice. No HDR-editing was detected in skeletal muscle and NHEJ-editing could not be quantified due to the high degree of multinucleation in this tissue. N=5 for experimental groups (N=2 mdx, N=3 C57BL/6J animals), N=3 for control groups (N=1 mdx, N=2 C57BL/6J animals).

FIGS. 5A-5D illustrate in vitro testing of GFP/BFP colour switching reporter system components. (FIG. 5A) show representative FACS plots showing that GFP and BFP can be distinguished by flow cytometry. mdx TTFs (no fluorescent protein) were transfected with plasmids of either CAG-GFP or CAG-BFP and analysed by flow cytometry 3 days later. (FIG. 5B) shows design of colour switching substitutions and GFPgRNAs. 2 base substitutions cause spectral shift and create a BtgI site for restriction fragment length polymorphism (RFLP) analysis. 3 SaCas9-compatible gRNAs targeting GFP near the substitution site were selected. GFPgRNA2 cuts closest to the desired colour-determining bases and recognition by this gRNA is disabled by HDR substitutions, which protects the 13H template and genomic HDR product from further Cas9 targeting. (FIG. 5C) show GFP disruption by GFPgRNAs. GFP^{+/-}; mdx TTFs were transfected with SaCas9 alone (control) or with SaCas9 plus one of the three gRNAs targeting GFP (see FIG. 5B). All three gRNAs disrupt GFP expression. GFPgRNA2 was selected for use in subsequent experiments due to its proximity to the colour switching mutations. GFPgRNA2 is referred to as GFPgRNA or gRNA in the main text. SSC, side scatter. (FIG. 5D) shows GFP disruption and lack of BFP expression in myoblasts transfected with SaCas9+GFPgRNA2, without BFP template. GFP^{+/-}; mdx myoblasts were transfected with lipofectamine only (lipo, control) or with SaCas9+GFPgRNA2, in the absence of the BFP template, and analysed by flow cytometry for GFP and BFP expression. GFP−/BFP− (CRISPR-NHEJ edited), but not BFP+, cells were present in cultures transfected with SaCas9 and gRNA, indicating that NHEJ alone is unable to induce green-to-blue spectral shift.

FIGS. 6A-6C illustrate differentiation and sequencing confirmation of ex vivo CRISPR-NHEJ and UDR edited myoblasts.(FIG. 6A) shows representative fluorescence images of myotubes differentiated from FACS sorted GFP+ (unedited), BFP+ (CRISPR-HDR edited) and GFP−/BFP− (CRISPR-NHEJ edited) myoblasts transfected previously with SaCas9 and GFPgRNA-BFP template. Scale bar, 100 um. Green, GFP; Blue, RFP; Red, myosin heavy chain (MHC). (FIG. 6B) shows restriction fragment length polymorphism (RFLP) analysis of genomic PCR products from .FACS sorted, culture expanded myoblasts. M, marker. (FIG. 6C) shows Sanger sequencing of genomic amplicons, aligned to GFP and BFP reference sequences confirms HDR in sorted BFP+ cells and NHEJ in sorted GIP−/BFP− cells.

FIG. 7 illustrates that systemic AAV-CRISPR enables in vivo CRISPR-NHEJ and CRISPR-HDR editing in myofibres of the tibialis anterior muscle of juvenile mdx animals. Representative fluorescence images for detection of CRISPR-NHEJ edited (GFP−/BFP−) and CRISPR-HDR edited (BFP+) cells in tibialis anterior muscles of mice receiving AAV-control (GFPgRNA-BFPtemplate only) or AAV-experimental (gRNA-temp+SaCas9). Each image is stitched together from 25 panels of 20× images. Scale bars, 200 um. Green, GFP; Blue, BFP; Red, Wheat Germ Agglutinin (WGA); white, TO-PRO-3.

FIGS. 8A-8B illustrate confirmation of CRISPR-NHEJ and HDR editing of skeletal muscle satellite cells in vivo by re-sorting of GFP+, GFP−/BFP− and BFP+ cells. (FIG. 8A) shows representative flow cytometric data showing analysis of GFP and BFP expression by skeletal muscle satellite cells isolated from juvenile mdx mice previously injected intravenously with vehicle AAV-GFPgRNA-BFPtemplate alone as control or AAV-GFPgRNA-BFPtemplate and AAV-SaCas9. Sort gates used for isolation of GFP+ (unedited), GFP−/BFP− (NHEJ-edited), and BFP+ (HDR-edited) cells are indicated. Sorted populations were expanded separately in culture for 2 weeks and then harvested for re-analysis (shown in FIG. 8B). (FIG. 8B) shows representative flow cytometric analysis of GFP and BFP expression. in culture expanded GFP−/BFP−, GFP+, and BFP+ cells previously sorted from AAV-HDR injected mice.

FIGS. 9A-9B illustrates that systemic AAV-CRISPR enables in vivo CRISPR-NHEJ and CRISPR-HDR editing in neonatal C57BL/6J animals. Representative fluorescence images for detection of CRISPR-NHEJ edited (GFP−/BFP−) and CRISPR-HDR edited (BFP+) cells in liver (shown in FIG. 9A) and cardiac muscle (shown in FIG. 9B) after intraperitoneal injection of AAV-GFPgRNA-BFPtemplate and AAV-SaCas9 (experimental) or AAV-GFPgRNA-BFPtemplate (control) to neonatal GFP^{+/-}; C57BL/6J mice. Scale bars, 50 um. Scale bars, 50 um. Green, GFP; Blue, BFP; Red, Wheat Germ Agglutinin (WGA); white. TO-PRO-3.

FIGS. 10A-10C illustrate Genomic PCR and Next-Generation Sequencing validation of in vivo CRISPR-NHEJ and. CRISPR-HDR editing. (FIG. 10A) shows schematics of the GFP/BFP genomic transgene loci and primers used for genomic PCR. Forward primer binds upstream of GFP/BFP start site on the genomic sequence, but not template DNA, and reverse primer binds downstream of Cas9 cutting site and colour switching substitutions. This primer pair amplifies the genomic transgene locus, but not the template sequence (due to absence of forward primer binding sequences in the template). (FIG. 10B) show representative aligned sequences from genomic NGS analysis of in vivo CRISPR-NHEJ and CRISPR-HDR edited satellite cells, TA muscle, heart, and liver of P21 AAV-HDR injected GFP^{+/-}; mdx mice. * representative NHEJ sequences shown; ** marks sites of insertions due to imprecise NHEJ. (FIG. 10C) shows read counts and allele frequencies (#unedited, HDR-edited, or NHEJ-edited reads/total reads mapped to the GFP/BFP sequence) of HDR− and NHEJ-edited alleles detected in satellite cells sorted from P21 GFP^{+/-}; mdx mice administered AAV-HDR or AAV-control in vivo. BFP⁺and GFP⁻/BFP⁻ cells were sorted from AAV-SaCas9 and AAV-gRNA-BFPtemplate injected experimental mice (AAV-HDR), and GFP⁺ cells were sorted from AAV-gRNA-BFPtemplate injected control mice (AAV-control).

FIGS. 11A-11C illustrate that satellite cells in neonatal skeletal muscles are infrequently targeted with systemic AAV-CR. SPR-HDR. (FIG. 11A) shows representative flow cytometric analysis of skeletal muscle satellite cells isolated from neonatal (P3) mdx and C57BL/6 mice 4 weeks after intraperitoneal injection with AAV-GFPgRNA-BFPtemplate alone as control or with AAV- GFPgRNA-BFPtemplate and AAV-SaCas9 to enable CRISPR-NHEJ and CRISPR-HDR. (FIG. 11B, 11C) show frequency (%) of CRISPR-HDR edited BFP+ satellite cells (FIG. 11B) and CRISPR-NHEJ edited GFP−/RFP− satellite cells (FIG. 11C), Individual data points are shown overlaid with mean±SD; AAV-Cas9 and AAV-gRNA-temp (experimental) N=2 mdx mice injected, N=3 C57BL6 mice injected; AAV-gRNA-temp only (control) N=1 mdx mouse injected, N=2 C57BL6 mice injected. *p<0.05, one-way analysis of variance (ANOVA) with Tukey's multiple comparisons test, DF=4.

FIG. 12 shows CRISPR-mediated editing results in a decrease of GFP fluorescence intensity in GFP mice in liver, heart, and tibialis anterior in mice treated at 3 days old (P3) or 21 days old (P21).

FIG. 13 shows CRISPR-mediated editing results in BPP fluorescence and decreased GFP fluorescence intensity in AAV-CRISPR injected P21 (21 day old mice at time of treatment) tibialis anterior. For each histogram, n=1400. Individual muscle fibers were circled as separate regions of interest in ImageJ, and mean fluorescence intensity in each fiber was measured using the “Measure” function. Histograms generated using Prism 8. Medians compared using Mann-Whitney U test.

FIG. 14 shows Sublaminar mononuclear cells in HDR-edited muscle are BFP+. Satellite cells are defined as sublaminar mononuclear cells.

### DETAILED DESCRIPTION OF THE INVENTION

Herein described are methods for precise, targeted gene replacement by HDR in skeletal and cardiac muscles, both largely post-mitotic tissues that have been widely considered to be inaccessible by this approach. Specifically, we demonstrate significant in vivo HDR-editing in the postnatal heart via systemic AAV delivery of CRISPR/Cas9, and greatly improved HDR editing rates in skeletal muscle. The methods described herein also enable HDR-editing in tissue stem cells within their native niche, allowing for direct manipulation of stem cell genomes therapeutically and experimentally, without the need to isolate, expand or transplant these rare cells.

Methods of Modifying the Genomes of Muscle Cells

Some aspects of the disclosure are directed to a method of modifying the genome of a muscle precursor cell in vivo in a subject, comprising contacting the muscle cell with one or more viruses, wherein the one or more viruses transduce a nucleic acid sequence encoding a sequence-targeting nuclease in the muscle precursor cell, and transduce a donor template in the muscle precursor cell, wherein the modification comprises the insertion of a nucleotide sequence corresponding to a nucleotide sequence of the donor template (e.g., via homologous recombination with the donor sequence). Homologous recombination (HR) mediated repair (also termed homology-directed repair (HDR)) uses homologous donor DNA as a template to repair a double stranded DNA break. If the sequence of the donor DNA differs from the genomic sequence, this process leads to the introduction of sequence changes into the genome.

The phase “modification of the genome” as used herein encompasses the addition of a regulatory sequence or a nucleotide sequence encoding a gene product via homologous recombination (i.e., insertion of a nucleotide sequence corresponding to a nucleotide sequence of the donor template). In some embodiments, the modification comprises replacement of a genomic region associated with a disease or condition (e.g., a genetic mutation) with a non-pathological genomic region via homologous recombination. For example, in some embodiments the modification comprises replacement of a genomic region comprising a mutation with a wild-type or non-mutated genomic region. In some embodiments, the mutation comprises a substitution or deletion mutation. In some embodiments, the modification comprises insertion of a nucleotide sequence in the genome corresponding to a deleted portion of a deletion mutation via homologous recombination. In some embodiments, the modification of the genome comprises insertion and/or replacement of a genomic sequence via homologous recombination that modulates the expression, activity or stability of a gene product. In some embodiments, the modification of the genome comprises modification of both alleles of the subject. In some embodiments, the modification of the genome comprises modification of one allele of the subject. In some embodiments, the genome modification comprises modification of one or more genes associated with biological processes. In some embodiments, the biological processes comprise epigenetic regulation or proteostasis (e.g., autophagy, ubiquitin-proteasome, heat shock response, anti-oxidant response, unfolded protein response).

As used herein, a “subject” means a human or animal (e.g., a primate). Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient”, “individual” and “subject” are used interchangeably herein. Preferably, the subject is a mammal. The mammal can be a human, non-human primate, mouse, rat, dog, cat, horse, or cow, but are not limited to these examples. A subject can be male or female. A “subject” may be any vertebrate organism in various embodiments. A subject may be individual to whom an agent is administered, e.g., for experimental, diagnostic, and/or therapeutic purposes or from whom a sample is obtained or on whom a procedure is performed. In some embodiments, a human subject is between newborn and 6 months old. In some embodiments, a human subject is between 6 and 24 months old. In some embodiments, a human subject is between 2 and 6, 6 and 12, or 12 and 18 years old. In some embodiments a human subject is between 18 and 30, 30 and 50, 50 and 80, or greater than 80 years old. In some embodiments, the subject is at least about 5, 10, 20, 30, 40, 50, 60, 65, 70, 75, 80, 85, or 90 years of age. In some embodiments, the subject is less than about 5, 10, 20, 30, 40, 50, 60, 65, 70, 75, 80, 85, or 90 years of age. In some embodiments, a subject is an adult. For purposes hereof a human at least 18 years of age is considered an adult. In some embodiments, the subject is a juvenile (e.g., less than about 18, 12 or 6 years of age for a human subject). In some embodiments, the subject is not a juvenile (e.g., less than about 18, 12 or 6 years of age for a human subject). In some embodiments a subject is an embryo. In some embodiments a subject is a fetus. In certain embodiments an agent is administered to a pregnant female in order to treat or cause a biological effect on an embryo or fetus in utero.

In some embodiments, the subject has a disease or condition involving muscle tissue. In some embodiments, the subject has, or has been diagnosed with a muscular dystrophy. In some embodiments, the muscular dystrophy is selected from myotonic muscular dystrophy, Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, and Emery-Dreifuss muscular dystrophy. In some embodiments, the muscular dystrophy is Becker muscular dystrophy or Duchenne muscular dystrophy. In some embodiments, the methods disclosed herein are used to treat a disease or condition of the subject.

As used herein, “contacting” a cell with one or more viruses can comprise administration of the virus systemically (e.g., intravenously) or locally (e.g., intramuscular injection) into the subject. Alternatively, other routes of administration may be selected (e.g., oral, inhalation, intranasal, intratracheal, intraarterial, intraocular, intravenous, intramuscular, and other parental routes). The method of contacting is not limited and may be any suitable method available in the art.

In some embodiments, virus compositions can be formulated in dosage units to contain an amount of replication-defective virus that is in the range of about 1.0×10⁹ GC to about 1.0×10¹⁵ GC (to treat an average subject of 70 kg in body weight), and preferably 1.0×10¹² GC to 1.0×10¹⁴ GC for a human patient. Preferably, the dose of replication-defective virus in the formulation is 1.0×10⁹ GC, 5.0×10⁹ GC, 1.0×10¹⁰ GC, 5.0×10¹⁰ GC, 1.0×10¹¹ GC, 5.0×10¹¹ GC, 1.0×10¹² GC, 5.0×10¹² GC, or 1.0×10¹³ GC, 5.0×10¹³ GC, 1.0×10¹⁴ GC, 5.0×10¹⁴ GC, or 1.0×10¹⁵ GC.

In some embodiments, at least about 0.1%, 0.5%, 1%, 2%, 3%, 5%, 10%, 15%, 20%, 25%, 30%, 35%, 40%, 45%, 50% or more of the genomes of the muscle precursor cells or a subset thereof are modified. In some embodiments, at least about 0.1%, 0.5%, 1%, 2%, 3%, 5%, 10%, 15%, 20%, 25%, 30%, 35%, 40%, 45%, 50% or more of the genome of the muscle precursor cells or a subset thereof are modified via homologous recombination (e.g., a genomic sequence is replaced or inserted via homologous recombination). . In some embodiments, at least about 40% or more of the genome of the muscle precursor cells or a subset thereof are modified via homologous recombination (e.g., a genomic sequence is replaced or inserted via homologous recombination). In some embodiments, at least 1% of muscle precursor cells in the subject are modified to comprise an insertion of a nucleotide sequence corresponding to a nucleotide sequence of the donor template. In some embodiments, at least 1%, 2%, 3%, 5%, 10%, 15%, 20%, 25%, 30%, 35%, 40%, 45%, 50% or more of muscle precursor cells in the subject are modified to comprise an insertion of a nucleotide sequence corresponding to a nucleotide sequence of the donor template. In some embodiments, the modification comprises a modification of at least one allele. In some embodiments, the modification comprises modification of both alleles.

Suitable viruses for use in the methods disclosed throughout the specification include, e.g., adenoviruses, adeno-associated viruses, retroviruses (e.g., lentiviruses), vaccinia virus and other poxviruses, herpesviruses (e.g., herpes simplex virus), and others. The virus may or may not contain sufficient viral genetic information for production of infectious virus when introduced into host cells, i.e., viral vectors may be replication-competent or replication-defective.

In some embodiments, the virus is adeno-associated virus. Adeno-associated virus (AAV) is a small (20 nm) replication-defective, nonenveloped virus. The AAV genome a single-stranded DNA (ssDNA) about 4.7 kilobase long. The genome comprises inverted terminal repeats (ITRs) at both ends of the DNA strand, and two open reading frames (ORFs): rep and cap. The AAV genome integrates most frequently into a particular site on chromosome 19. Random incorporations into the genome take place with a negligible frequency. The integrative capacity may be eliminated by removing at least part of the rep ORF from the vector resulting in vectors that remain episomal and provide sustained expression at least in non-dividing cells. To use AAV as a gene transfer vector, a nucleic acid comprising a nucleic acid sequence encoding a desired protein or RNA, e.g., encoding a polypeptide or RNA that inhibits ATPIF1 , operably linked to a promoter, is inserted between the inverted terminal repeats (ITR) of the AAV genome. Adeno-associated viruses (AAV) and their use as vectors, e.g., for gene therapy, are also discussed in Snyder, RO and Moullier, P., Adeno-Associated Virus Methods and Protocols, Methods in Molecular Biology, Vol. 807. Humana Press, 2011.

In some embodiments, the AAV is AAV serotype 6, 8, 9, 10 or Anc80 (disclosed in WO2015054653, incorporated herein by reference). In some embodiments, the AAV serotype is AAV serotype 2. Any AAV serotype, or modified AAV serotype, may be used as appropriate and is not limited.

Another suitable AAV may be, e.g., rh1O [see, e.g., WO 2003/042397]. Still other AAV sources may include, e.g., AAV9 [see, e.g., U.S. Pat. No. 7,906,111; US 2011-0236353-A1], and/or hu37 [see, e.g., U.S. Pat. No. 7,906,111; US 2011-0236353-A1], AAV1, AAV2, AAV3, AAV4, AAVS, AAV6, AAV6.2, AAV7, AAV8, [see, e.g., U.S. Pat. No. 7,790,449; U.S. Pat. No. 7,282,199] and others. See, e.g., WO 2003/042397; WO 2005/033321, WO 2006/110689; U.S. Pat. Nos. 7,790,449; 7,282,199; and 7,588,772 B2 for sequences of these and other suitable AAV, as well as for methods for generating AAV vectors. Still other AAV may be selected, optionally taking into consideration tissue preferences of the selected AAV capsid. A recombinant AAV vector (AAV viral particle) may comprise, packaged within an AAV capsid, a nucleic acid molecule containing a 5′ AAV ITR, the expression cassettes described herein and a 3′ AAV ITR. As described herein, an expression cassette may contain regulatory elements for an open reading frame(s) within each expression cassette and the nucleic acid molecule may optionally contain additional regulatory elements.

The AAV vector may contain a full-length AAV 5′ inverted terminal repeat (ITR) and a full-length 3′ ITR. A shortened version of the 5′ ITR, termed AITR, has been described in which the D-sequence and terminal resolution site (trs) are deleted. The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

Where a pseudotyped AAV is to be produced, the ITRs are selected from a source which differs from the AAV source of the capsid. For example, AAV2 ITRs may be selected for use with an AAV capsid having a particular efficiency for a selected cellular receptor, target tissue or viral target. In one embodiment, the ITR sequences from AAV2, or the deleted version thereof (AITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. However, other sources of AAV ITRs may be utilized.

A single-stranded AAV viral vector may be used. Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2. In one system, a producer cell line is transiently transfected with a construct that encodes the transgene flanked by ITRs and a construct(s) that encodes rep and cap. In a second system, a packaging cell line that stably supplies rep and cap is transfected (transiently or stably) with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus ULS, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al, 2009, “Adenovirus- adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

In another embodiment, other viral vectors may be used, including integrating viruses, e.g., herpesvirus or lentivirus, although other viruses may be selected. Suitably, where one of these other vectors is generated, it is produced as a replication-defective viral vector. A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production.

The one or more viruses may contain a promoter capable of directing expression (e.g., expression of a sequence-targeting nuclease, donor template, and/or one or more gRNAs) in mammalian cells, such as a suitable viral promoter, e.g., from a cytomegalovirus (CMV), retrovirus, simian virus (e.g., SV40), papilloma virus, herpes virus or other virus that infects mammalian cells, or a mammalian promoter from, e.g., a gene such as EF1alpha, ubiquitin (e.g., ubiquitin B or C), globin, actin, phosphoglycerate kinase (PGK), etc., or a composite promoter such as a CAG promoter (combination of the CMV early enhancer element and chicken beta-actin promoter). In some embodiments a human promoter may be used. In some embodiments, the promoter is selected from a CMV promoter, U6 promoter, an H1 promoter, a constitutive promoter, and a ubiquitous promoter. In some embodiments, the promoter directs expression in a particular cell type. For example, a muscle precursor cell specific promoter.

In some embodiments of each of the methods disclosed herein, a suitable tissue specific promoter can be obtained by a person of ordinary skill in the art from the tissue specific promoters set forth in “TiProD: Tissue specific promoter Database” available on the world-wide web at tiprod.bioinf.med.uni-goettingen.de/.

The sequence-targeting nucleases that can be used in the methods disclosed herein are not limited and may be any sequence-targeting nucleases disclosed herein. In some embodiments, the sequence-targeting nuclease is a Zinc-Finger Nuclease (ZFN), a Transcription activator-like effector nuclease (TALEN), a Cas nuclease (e.g., Cas9 nuclease), or a functional fragment or functional variant thereof.

There are currently four main types of sequence-targeting nucleases (i.e., targetable nucleases, site specific nucleases) in use: zinc finger nucleases (ZFNs), transcription activator—like effector nucleases (TALENs), and RNA-guided nucleases (RGNs) such as the Cas proteins of the CRISPR/Cas Type II system, and engineered meganucleases. ZFNs and TALENs comprise the nuclease domain of the restriction enzyme FokI (or an engineered variant thereof) fused to a site-specific DNA binding domain (DBD) that is appropriately designed to target the protein to a selected DNA sequence. In the case of ZFNs, the DNA binding domain (DBD) comprises a zinc finger DBD. In the case of TALENs, the site-specific DBD is designed based on the DNA recognition code employed by transcription activator—like effectors (TALEs), a family of site-specific DNA binding proteins found in plant-pathogenic bacteria such as Xanthomonas species.

The Clustered Regularly Interspaced Short Palindromic Repeats (CRISPR) Type II system is a bacterial adaptive immune system that has been modified for use as an RNA-guided endonuclease technology for genome engineering. The bacterial system comprises two endogenous bacterial RNAs called crRNA and tracrRNA and a CRISPR-associated (Cas) nuclease, e.g., Cas9. The tracrRNA has partial complementarity to the crRNA and forms a complex with it. The Cas protein is guided to the target sequence by the crRNA/tracrRNA complex, which forms a RNA/DNA hybrid between the crRNA sequence and the complementary sequence in the target. For use in genome modification, the crRNA and tracrRNA components are often combined into a single chimeric guide RNA (sgRNA or gRNA) in which the targeting specificity of the crRNA and the properties of the tracrRNA are combined into a single transcript that localizes the Cas protein to the target sequence so that the Cas protein can cleave the DNA. The sgRNA often comprises an approximately 20 nucleotide guide sequence complementary or homologous to the desired target sequence followed by about 80 nt of hybrid crRNA/tracrRNA. One of ordinary skill in the art appreciates that the guide RNA need not be perfectly complementary or homologous to the target sequence. For example, in some embodiments it may have one or two mismatches. The genomic sequence which the gRNA hybridizes is typically flanked on one side by a Protospacer Adjacent Motif (PAM) sequence although one of ordinary skill in the art appreciates that certain Cas proteins may have a relaxed requirement for a PAM sequence. The PAM sequence is present in the genomic DNA but not in the sgRNA sequence. The Cas protein will be directed to any DNA sequence with the correct target sequence and PAM sequence. The PAM sequence varies depending on the species of bacteria from which the Cas protein was derived. Specific examples of Cas proteins include Cas1, Cas2, Cas3, Cas4, Cas5, Cas6, Cas7, Cas8, Cas9 and Cas10. In some embodiments, the site specific nuclease comprises a Cas9 protein. For example, Cas9 from Streptococcus pyogenes (Sp), Neisseria meningitides, Staphylococcus aureus, Streptococcus thermophiles, or Treponema denticola may be used. The PAM sequences for these Cas9 proteins are NGG, NNNNGATT, NNAGAA, NAAAAC, respectively. In some embodiments, the Cas9 is from Staphylococcus aureus (saCas9).

A number of engineered variants of the site-specific nucleases have been developed and may be used in certain embodiments. For example, engineered variants of Cas9 and Fok1 are known in the art. Furthermore, it will be understood that a biologically active fragment or variant can be used. Other variations include the use of hybrid site specific nucleases. For example, in CRISPR RNA-guided FokI nucleases (RFNs) the Fok1 nuclease domain is fused to the amino-terminal end of a catalytically inactive Cas9 protein (dCas9) protein. RFNs act as dimers and utilize two guide RNAs (Tsai, QS, et al., Nat Biotechnol. 2014; 32(6): 569-576). Site-specific nucleases that produce a single-stranded DNA break are also of use for genome editing. Such nucleases, sometimes termed “nickases” can be generated by introducing a mutation (e.g., an alanine substitution) at key catalytic residues in one of the two nuclease domains of a site specific nuclease that comprises two nuclease domains (such as ZFNs, TALENs, and Cas proteins). Examples of such mutations include D10A, N863A, and H840A in SpCas9 or at homologous positions in other Cas9 proteins. A nick can stimulate HDR at low efficiency in some cell types. Two nickases, targeted to a pair of sequences that are near each other and on opposite strands can create a single-stranded break on each strand (“double nicking”), effectively generating a DSB, which can optionally be repaired by HDR using a donor DNA template (Ran, F. A. et al. Cell 154, 1380-1389 (2013). In some embodiments, the Cas protein is a SpCas9 variant. In some embodiments, the SpCas9 variant is a R661A/Q695A/Q926A triple variant or a N497A/R661A/Q695A/Q926A quadruple variant. See Kleinstiver et al., “High-fidelity CRISPR-Cas9 nucleases with no detectable genome-wide off-target effects,” Nature, Vol. 529, pp. 490-495 (and supplementary materials)(2016); incorporated herein by reference in its entirety. In some embodiments, the Cas protein is C2c1, a class 2 type V-B CRISPR-Cas protein. See Yang et al., “PAM-Dependent Target DNA Recognition and Cleavage by C2c1 CRISPR-Cas Endonuclease,” Cell, Vol. 167, pp. 1814-1828 (2016); incorporated herein by reference in its entirety. In some embodiments, the Cas protein is one described in US 20160319260 “Engineered CRISPR-Cas9 nucleases with Altered PAM Specificity” incorporated herein by reference.

The nucleic acid encoding the sequence-targeting nuclease should be sufficiently short to be included in the virus (e.g., AAV). In some embodiments, the nucleic acid encoding the sequence-targeting nuclease is less than 4.4. kb.

In some embodiments, the sequence-targeting nuclease has at least about 80%, 85%, 90%, 91%, 92%, 93%, 94%, 95%, 96%, 97%, 98% or 99% polypeptide sequence identity to a naturally occurring targetable nuclease.

In some embodiments, the one or more viruses comprise a first virus which transduces a nucleic acid sequence encoding a sequence-targeting nuclease and a donor template. In some embodiments, the one or more viruses comprise a first virus which transduces a nucleic acid sequence encoding a sequence-targeting nuclease, a donor template and one or more (e.g, one, two, three, four, etc.) gRNAs. In embodiments of the methods described herein wherein a single virus transduces the sequence-targeting nuclease, the donor template, and, optionally, one or more gRNAs a person of ordinary skill in the art can select a suitable virus capable of packaging the required nucleotide sequences. In some embodiments, the one or more viruses comprise a first virus which transduces a nucleic acid sequence encoding a sequence-targeting nuclease, and a second virus which transduces a donor template. In some embodiments, the one or more viruses comprise a first virus which transduces a nucleic acid sequence encoding a sequence-targeting nuclease, and a second virus which transduces a donor template and one or more (e.g, one, two, three, four, etc.) gRNAs. In some embodiments, the one or more viruses comprise a first virus which transduces a nucleic acid sequence encoding a sequence-targeting nuclease, and a second virus which transduces a donor template and two gRNAs. In some embodiment, the ratio of the first virus to the second virus is about 1:3 to about 1:100, inclusive of intervening ratios. For example, the ratio of the first virus to the second virus may be about 1:5 to about 1:50, or about 1:10, or about 1:20. Although not as preferred, the ratio may be 1:1 or there may be more second virus.

In some embodiments, the method comprises delivery of one or more components (e.g., nucleic acid encoding a sequence-targeting nuclease, a donor template, one or more gRNAs (e.g., two gRNAs)) mediated by non-viral constructs, e.g. , “naked DNA”, “naked plasmid DNA”, RNA, and mRNA; coupled with various delivery compositions and nanoparticles, including, e.g., micelles, liposomes, cationic lipid-nucleic acid compositions, poly-glycan compositions and other polymers, lipid and/or cholesterol-based-nucleic acid conjugates, and other constructs such as are described herein. See, e.g., X. Su et al, Mol. Pharmaceutics, 201 1, 8 (3), pp 774-787; web publication: Mar. 21, 2011; WO2013/182683, WO 2010/053572 and WO 2012/170930, all of which are incorporated herein by reference.

In some embodiments, the muscle precursor cell having its genome modified by the methods disclosed herein is a muscle stem cell (e.g., adult muscle stem cell). However, the muscle precursor cell is not limited. In some embodiments, at least 1% of muscle precursor cells (e.g., muscle stem cells) in the subject are modified to comprise an insertion of a nucleotide sequence corresponding to a nucleotide sequence of the donor template. In other embodiments of the invention, the methods disclosed herein comprise modification of myofibre cells. In some embodiments, both muscle precursor cells and myofibre cells have their genomes modified. In some embodiments, the genomes of myofibre cells are not, or are not substantially, modified.

Some aspects of the invention are directed to methods of making myofibres with modified genomes by modifying the genomes of muscle precursor cells (e.g., satellite cells) by the methods disclosed herein. The modified myofibres comprise one or more modified muscle precursor cell nuclei. In some embodiments, the myofibres comprise at least one, two, three, four, five, ten, twenty, fifty, seventy-five, one hundred, two hundred, two hundred fifty, three hundred, four hundred or more modified nuclei. In some embodiments, at least about 1%, 2%, 3%, 5%, 10%, 20%, 30%, 40%, 50%, 51%, 60%, 70%, 90%, 95%, or 99% of the nuclei of a myofibre have genomes modified by the methods disclosed herein. In some embodiments, at least about 1%, 2%, 3%, 5%, 10%, 20%, 30%, 40%, 50%, 51%, 60%, 70%, 90%, 95%, or 99% of the myofibres of the subject have genomes modified by the methods disclosed herein. In some embodiments, the subject having myofibres modified by the methods disclosed herein has been diagnosed with a muscular dystrophy. In some embodiments, the subject has a muscular dystrophy. In some embodiments, the muscular dystrophy is selected from myotonic muscular dystrophy, Duchenne muscular dystrophy (DMD), Becker muscular dystrophy, limb-girdle muscular dystrophy, facioscapulohumeral muscular dystrophy, congenital muscular dystrophy, oculopharyngeal muscular dystrophy, distal muscular dystrophy, and Emery-Dreifuss muscular dystrophy. In some embodiments, the muscular dystrophy is Becker muscular dystrophy or Duchenne muscular dystrophy.

In some embodiments, the methods disclosed herein further comprise assessing the fate or function of muscle progenitor cells or myofibres with genomes modified by the methods disclosed herein.

Methods of Modifying the Genome of Cardiac Cells

Some aspects of the disclosure are directed to methods of modifying the genome of a cardiac cell in vivo in a subject, comprising contacting the cardiac cell with one or more viruses, wherein the one or more viruses transduce a nucleic acid sequence encoding a sequence-targeting nuclease in the cardiac cell, and transduce a donor template in the cardiac cell, wherein the modification comprises the insertion of a nucleotide sequence corresponding to a nucleotide sequence of the donor template (e.g, homologous recombination), and wherein the cardiac cell is a DNA synthesizing cardiac cell or a replicating cardiac cell.

The subject is not limited and may be any subject as described herein. In some embodiments, the subject has a cardiac disease or condition. In some embodiments, the cardiac disease or condition is associated with a genetic mutation. In some embodiments, the cardiac disease or condition can be ameliorated or treated by correcting a genetic mutation. In some embodiments, the cardiac disease or condition can be ameliorated or treated by insertion of a genetic sequence into the genomes of cardiac cells. In some embodiments, the likelihood of a cardiac disease or condition can be reduced or prevented by correction of a genetic mutation. In some embodiments, the likelihood of a cardiac disease or condition can be reduced or prevented by insertion of a genetic sequence into the genomes of cardiac cells. In some embodiments, the subject is an infant, or juvenile, or under 30 years of age. In some embodiments, the subject is not an infant, or juvenile, or under 30 years of age.

In some embodiments, the cardiac cell is selected from the group consisting of a mammalian postmitotic cardiomyocyte, a mammalian postmitotic cardiomyocyte capable of DNA synthesis without division/proliferation, a human postmitotic cardiomyocyte, a human postmitotic cardiomyocyte capable of DNA synthesis without division/proliferation, a cardiomyocyte precursor cell, a proliferating mesenchymal cardiac cell, a proliferating endothelial cardiac cell, and a cardiac progenitor cell.

The sequence-targeting nuclease is not limited and may be any sequence-targeting nuclease described herein. In some embodiments, the sequence-targeting nuclease is Cas9 or a functional fragment or functional variant thereof.

In some embodiments, the one or more viruses comprise a first virus which transduces a nucleic acid sequence encoding a sequence-targeting nuclease and a donor template. In some embodiments, the one or more viruses comprise a first virus which transduces a nucleic acid sequence encoding a sequence-targeting nuclease, a donor template and one or more (e.g, one, two, three, four, etc.) gRNAs. In embodiments of the methods described herein wherein a single virus transduces the sequence-targeting nuclease, the donor template, and, optionally, one or more gRNAs a person of ordinary skill in the art can select a suitable virus capable of packaging the required nucleotide sequences. In some embodiments, the one or more viruses comprise a first virus which transduces a nucleic acid sequence encoding a sequence-targeting nuclease, and a second virus which transduces a donor template. In some embodiments, the one or more viruses comprise a first virus which transduces a nucleic acid sequence encoding a sequence-targeting nuclease, and a second virus which transduces a donor template and one or more (e.g, one, two, three, four, etc.) gRNAs. In some embodiment, the ratio of the first virus to the second virus is about 1:3 to about 1:100, inclusive of intervening ratios. For example, the ratio of the first virus to the second virus may be about 1:5 to about 1:50, or about 1:10, or about 1:20. Although not as preferred, the ratio may be 1:1 or there may be more second virus.

In some embodiments, the method comprises delivery of one or more components (e.g., nucleic acid encoding a sequence-targeting nuclease, a donor template, one or more gRNAs) mediated by non-viral constructs, e.g. , “naked DNA”, “naked plasmid DNA”, RNA, and mRNA; coupled with various delivery compositions and nanoparticles, including, e.g., micelles, liposomes, cationic lipid-nucleic acid compositions, poly-glycan compositions and other polymers, lipid and/or cholesterol-based-nucleic acid conjugates, and other constructs such as are described herein. See, e.g., X. Su et al, Mol. Pharmaceutics, 201 1, 8 (3), pp 774-787; web publication: Mar. 21, 2011 ; WO2013/182683, WO 2010/053572 and WO 2012/170930, both of which are incorporated herein by reference.

The one or more viruses may contain a promoter capable of directing expression (e.g., expression of a sequence-targeting nuclease, donor template, one or more gRNA) in mammalian cells, such as a suitable viral promoter as described herein. In some embodiments a human promoter may be used. In some embodiments, the promoter is selected from a CMV promoter, U6 promoter, an H1 promoter, a constitutive promoter, and a ubiquitous promoter. In some embodiments, the promoter directs expression in a particular cell type. For example, in some embodiments, the promoter is a cardiac specific promoter (e.g., a mammalian postmitotic cardiomyocyte specific promoter, a mammalian postmitotic cardiomyocyte capable of DNA synthesis without division/proliferation specific promoter, a human postmitotic cardiomyocyte specific promoter, a human postmitotic cardiomyocyte capable of DNA synthesis without division/proliferation specific promoter, a cardiomyocyte precursor cell specific promoter, a proliferating mesenchymal cardiac cell specific promoter, a proliferating endothelial cardiac cell specific promoter, or a cardiac progenitor cell specific promoter, or a promoter specific to one or more of these listed subtypes). In some embodiments, the nucleic acid sequence encoding a sequence-targeting nuclease is transduced with a cardiac specific promoter, a ubiquitous promoter or a non-specific promoter.

The one or more viruses used are not limited and may be any suitable virus or virus disclosed herein. In some embodiments, the virus is AAV serotype 6, 8, 9, 10 or Anc80.

In some embodiments, virus compositions can be formulated in dosage units to contain an amount of replication-defective virus that is in the range of about 1.0×10⁹ GC (genomic copies, also referred to herein as viral genomes (vg)) to about 1.0×10¹⁵ GC (to treat an average subject of 70 kg in body weight), and preferably 1.0×10¹² GC to 1.0×10¹⁴ GC for a human patient. Preferably, the dose of replication-defective virus in the formulation is 1.0×10⁹ GC, 5.0×10⁹ GC, 1.0×10¹⁰ GC, 5.0×10¹⁰ GC, 1.0×10¹¹ GC, 5.0×10¹¹ GC, 1.0×10¹² GC, 5.0×10¹² GC, or 1.0×10¹³ GC, 5.0×10¹³ GC, 1.0×10¹⁴ GC, 5.0×10¹⁴ GC, or 1.0×10¹⁵ GC.

In some embodiments, at least about 0.1%, 0.5%, 1%, 2%, 3%, 5%, 10%, 15%, 20%, 25%, 30%, 35%, 40%, 45%, 50% or more of the genomes of the cardiac cells of the subject are modified. In some embodiments, at least about 0.1%, 0.5%, 1%, 2%, 3%, 5%, 10%, 15%, 20%, 25%, 30%, 35%, 40%, 45%, 50% or more of the genome of the cardiac cells are modified via homologous recombination (e.g., a genomic sequence is replaced or inserted via homologous recombination). In some embodiments, at least 1%, 1.6%, 2% of cardiac cells in the subject are modified to comprise an insertion of a nucleotide sequence corresponding to a nucleotide sequence of the donor template. In some embodiments, the modification comprises a modification of at least one allele. In some embodiments, the modification comprises modification of both alleles.

Some aspects of the disclosure are directed to cardiac tissue comprising cardiac cells with genomes modified by methods disclosed herein. In some embodiments, the cardiac tissue comprises progeny cells of cardiac cells modified by the methods disclosed herein. In some embodiments, at least about 1%, 2%, 3%, 5%, 10%, 20%, 30%, 40%, 50%, 51%, 60%, 70%, 90%, 95%, 99% of the muscle cells of the cardiac tissue have been modified or are progeny of cells that have been modified by the methods disclosed herein. In some embodiments, the subject having cardiac tissue modified by the methods disclosed herein has been diagnosed with a cardiac disease or condition. In some embodiments, the cardiac condition is damaged cardiac muscle (e.g. cardiac muscle damaged followed myocardial infarction). In some embodiments, the cardiac disease is myocardial infarction, ischemic heart disease, dilated cardiomyopathy, heart failure (e.g., congestive heart failure), ischemic cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis, aortic stenosis, aortic regurgitation, mitral stenosis, mitral regurgitation, mitral prolapse, pulmonary stenosis, pulmonary stenosis, pulmonary regurgitation, tricuspid stenosis, tricuspid regurgitation, congenital disorder, genetic disorder, or a combination thereof. In some embodiments, the methods disclosed herein can be utilized to promote cardiac muscle regeneration in a subject in need thereof.

In some embodiments, the methods disclosed herein further comprise assessing the fate or function of cardiac cells with genome modification.

Methods of Targeting Specific Striated Muscles Types for Genomic Modification

Some aspects of the disclosure are directed to methods of targeting a specific striated muscle type for genomic modification in vivo in a subject via homology directed repair, comprising systemically administering with one or more viruses, wherein the one or more viruses transduce a nucleic acid sequence encoding a sequence-targeting nuclease in striated muscle cells, and transduce a donor template in striated muscle cells, wherein the modification comprises the insertion of a nucleotide sequence corresponding to a nucleotide sequence of the donor template, and wherein, due to the age of the subject, genomic modification preferentially occurs to at least one type of striated muscle.

In some embodiments, the genomes of muscle precursor cells are preferentially modified. In some embodiments, the genomes of cardiac cells are preferentially modified.

The subject is not limited and may be any subject as described herein. In some embodiments, the subject has a muscle or cardiac disease or condition.

The sequence-targeting nuclease is not limited and may be any sequence-targeting nuclease described herein. In some embodiments, the sequence-targeting nuclease is Cas9 or a functional fragment or functional variant thereof.

In some embodiments, the one or more viruses comprise a first virus which transduces a nucleic acid sequence encoding a sequence-targeting nuclease and a donor template. In some embodiments, the one or more viruses comprise a first virus which transduces a nucleic acid sequence encoding a sequence-targeting nuclease, a donor template and one or more (e.g, one, two, three, four, etc.) gRNAs. In embodiments of the methods described herein wherein a single virus transduces the sequence-targeting nuclease, the donor template, and, optionally, one or more gRNAs a person of ordinary skill in the art can select a suitable virus capable of packaging the required nucleotide sequences. In some embodiments, the one or more viruses comprise a first virus which transduces a nucleic acid sequence encoding a sequence-targeting nuclease, and a second virus which transduces a donor template. In some embodiments, the one or more viruses comprise a first virus which transduces a nucleic acid sequence encoding a sequence-targeting nuclease, and a second virus which transduces a donor template and one or more (e.g, one, two, three, four, etc.) gRNAs. In some embodiment, the ratio of the first virus to the second virus is about 1:3 to about 1:100, inclusive of intervening ratios. For example, the ratio of the first virus to the second virus may be about 1:5 to about 1:50, or about 1:10, or about 1:20. Although not as preferred, the ratio may be 1:1 or there may be more second virus.

In some embodiments, the method comprises delivery of one or more components (e.g., nucleic acid encoding a sequence-targeting nuclease, a donor template, one or more gRNAs) mediated by non-viral constructs, e.g. , “naked DNA”, “naked plasmid DNA”, RNA, and mRNA; coupled with various delivery compositions and nanoparticles, including, e.g., micelles, liposomes, cationic lipid-nucleic acid compositions, poly-glycan compositions and other polymers, lipid and/or cholesterol-based-nucleic acid conjugates, and other constructs such as are described herein. See, e.g., X. Su et al, Mol. Pharmaceutics, 201 1, 8 (3), pp 774-787; web publication: Mar. 21, 2011 ; WO2013/182683, WO 2010/053572 and WO 2012/170930, both of which are incorporated herein by reference.

The one or more viruses may contain a promoter capable of directing expression (e.g., expression of a sequence-targeting nuclease, donor template, one or more gRNA) in mammalian cells, such as a suitable viral promoter as described herein. In some embodiments a human promoter may be used. In some embodiments, the promoter is selected from a CMV promoter, U6 promoter, an H1 promoter, a constitutive promoter, and a ubiquitous promoter. In some embodiments, the promoter directs expression in a particular cell type. In some embodiments, the nucleic acid sequence encoding a sequence-targeting nuclease is transduced with a ubiquitous promoter or a non-specific promoter.

The one or more viruses used are not limited and may be any suitable virus or virus disclosed herein. In some embodiments, the virus is AAV serotype 6, 8, 9, 10 or Anc80.

In some embodiments, virus compositions can be formulated in dosage units to contain an amount of replication-defective virus that is in the range of about 1.0×10⁹ GC to about 1.0×10¹⁵ GC (to treat an average subject of 70 kg in body weight), and preferably 1.0×10¹² GC to 1.0×10¹⁴ GC for a human patient. Preferably, the dose of replication-defective virus in the formulation is 1.0×10⁹ GC, 5.0×10⁹ GC, 1.0×10¹⁰ GC, 5.0×10¹⁰ GC, 1.0×10¹¹ GC, 5.0×10¹¹ GC, 1.0×10¹² GC, 5.0×10¹² GC, or 1.0×10¹³ GC, 5.0×10¹³ GC, 1.0×10¹⁴ GC, 5.0×10¹⁴ GC, or 1.0×10¹⁵ GC.

In some embodiments, at least about 0.1%, 0.5%, 1%, 2%, 3%, 5%, 10%, 15%, 20%, 25%, 30%, 35%, 40%, 45%, 50% or more of the genomes of a striated muscle cell type (e.g., cardiac muscle, muscle progenitor cell, myofibre) of the subject are modified. In some embodiments, at least about 0.1%, 0.5%, 1%, 2%, 3%, 5%, 10%, 15%, 20%, 25%, 30%, 35%, 40%, 45%, 50% or more of the genomes of a striated muscle cell type (e.g., cardiac muscle, muscle progenitor cell, myofibre) are modified via homologous recombination (e.g., a genomic sequence is replaced or inserted via homologous recombination). In some embodiments, at least about 1%, 1.6%, or 2% of a striated muscle cell type (e.g., cardiac muscle, muscle progenitor cell, myofibre, etc.) in the subject are modified to comprise an insertion of a nucleotide sequence corresponding to a nucleotide sequence of the donor template. In some embodiments, the modification comprises a modification of at least one allele. In some embodiments, the modification comprises modification of both alleles.

In some embodiments, a human subject is between 6 and 24 months old. In some embodiments, a human subject is between 2 and 6, 6 and 12, or 12 and 18 years old. In some embodiments a human subject is between 18 and 30, 30 and 50, 50 and 80, or greater than 80 years old. In some embodiments, the subject is at least about 5, 10, 20, 30, 40, 50, 60, 65, 70, 75, 80, 85, or 90 years of age. In some embodiments, the subject is less than about 5, 10, 20, 30, 40, 50, 60, 65, 70, 75, 80, 85, or 90 years of age. In some embodiments, a subject is an adult. For purposes hereof a human at least 18 years of age is considered an adult. In some embodiments, the subject is a juvenile (e.g., less than about 18, 12 or 6 years of age for a human subject). In some embodiments, the subject is not a juvenile (e.g., less than about 18, 12 or 6 years of age for a human subject). In some embodiments, the subject is less than 1 year of age. In some embodiments, the subject is more than 1 year of age and less than 6 years of age. In some embodiments, the subject is more than 6 years of age and less than 12 years of age. In some embodiments, the subject is more than 12 years of age and less than 18 years of age. In some embodiments, the subject is more than 18 years of age and less than 24 years of age. In some embodiments, the subject is more than 18 years of age. In some embodiments, the subject is post-puberty. In some embodiments, the subject is pre-puberty. In some embodiments, the subject is undergoing puberty. In some embodiments a subject is an embryo. In some embodiments a subject is a fetus. In certain embodiments an agent is administered to a pregnant female in order to treat or cause a biological effect on an embryo or fetus in utero.

In some embodiments, the methods disclosed herein further comprise assessing the fate or function of striated muscle cells with genome modification.

The terms “decrease,” “reduce,” “reduced,” “reduction,” “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , up to and including, for example, the complete absence of the given entity or parameter as compared to the reference level, or any decrease between 10-99% as compared to the absence of a given treatment.

The terms “increased,” “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90%, or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or more as compared to a reference level.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The term “statistically significant” or “significantly” refers to statistical significance and generally means a “p” value greater than 0.05 (calculated by the relevant statistical test). Those skilled in the art will readily appreciate that the relevant statistical test for any particular experiment depends on the type of data being analyzed. Additional definitions are provided in the text of individual sections below.

Definitions of common terms in cell biology and molecular biology can be found in “The Merck Manual of Diagnosis and Therapy”, 19th Edition, published by Merck Research Laboratories, 2006 (ISBN 0-911910-19-0); RobertS. Porter et al. (eds.), The Encyclopedia of Molecular Biology, published by Blackwell Science Ltd., 1994 (ISBN 0-632-02182-9); The ELISA guidebook (Methods in molecular biology 149) by Crowther J. R. (2000); Immunology by Werner Luttmann, published by Elsevier, 2006. Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Cun-ent Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001) and Davis et al., Basic Methods in Molecular Biology, Elsevier Science Publishing, Inc., New York, USA (1995) which are both incorporated by reference herein in their entireties.

As used herein, the terms “proteins” and “polypeptides” are used interchangeably to designate a series of amino acid residues connected to the other by peptide bonds between the alpha-amino and carboxy groups of adjacent residues. The terms “protein”, and “polypeptide” refer to a polymer of protein amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when refining to a gene product and fragments thereof.

Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double stranded DNA. Alternatively, it can be a single-stranded nucleic acid not derived from any double stranded DNA. In one aspect, the template nucleic acid is DNA. In another aspect, the template is RNA. Suitable nucleic acid molecules are DNA, including genomic DNA or cDNA. Other suitable nucleic acid molecules are RNA, including mRNA. The nucleic acid molecule can be naturally occurring, as in genomic DNA, or it may be synthetic, i.e., prepared based upon human action, or may be a combination of the two. The nucleic acid molecule can also have certain modification such as 2′-deoxy, 2′-deoxy-2′fluoro, 2′-0-methyl, 2′-0-methoxyethyl (2′-0-MOE), 2′-0-aminopropyl (2′-0-AP), 2′-0-dimethylaminoethyl (2′-0-DMA0E), 2′-0-dimethylaminopropyl (2′-0-DMAP), 2′-0-dimethylaminoethyloxyethyl (2′-0-DMAEOE), or 2′-0-N-methylacetamido (2′-0-NMA), cholesterol addition, and phosphorothioate backbone as described in US Patent Application 20070213292; and certain ribonucleoside that are is linked between the 2′ -oxygen and the 4′ -carbon atoms with a methylene unit as described in U.S. Pat No. 6,268,490, wherein both patent and patent application are incorporated hereby reference in their entirety.

As used herein, “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state as compared to that expected in the absence of treatment.

The efficacy of a given treatment for a disorder or disease can be determined by the skilled clinician. However, a treatment is considered “effective treatment,” as the term is used herein, if any one or all of the signs or symptoms of a disorder are altered in a beneficial manner, other clinically accepted symptoms are improved or ameliorated, e.g., by at least 10% following treatment with an agent or composition as described herein. Efficacy can also be measured by a failure of an individual to worsen as assessed by hospitalization or need for medical interventions (i.e., progression of the disease is halted). Methods of measuring these indicators are known to those of skill in the art and/or described herein.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

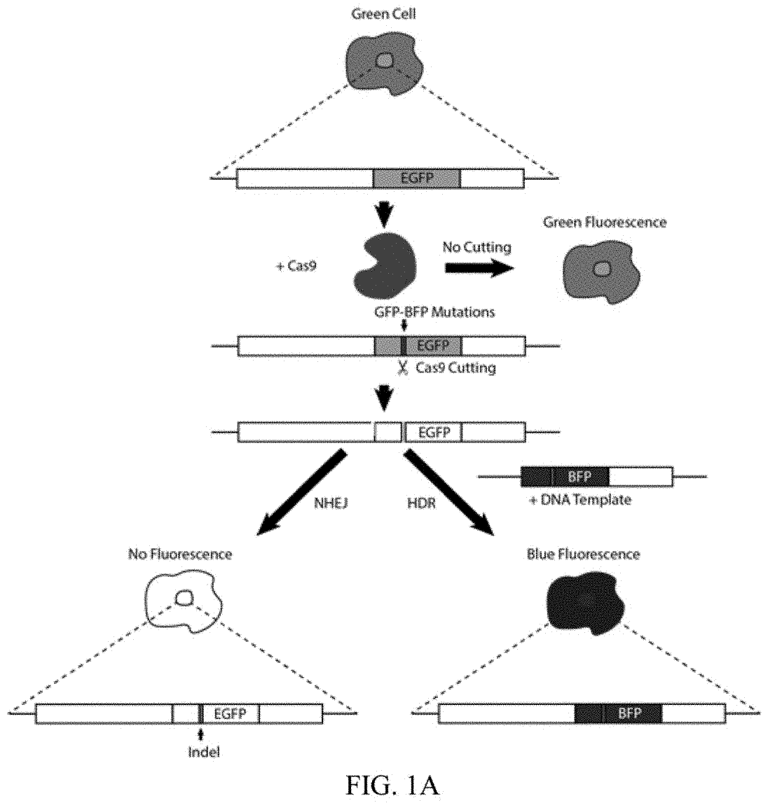

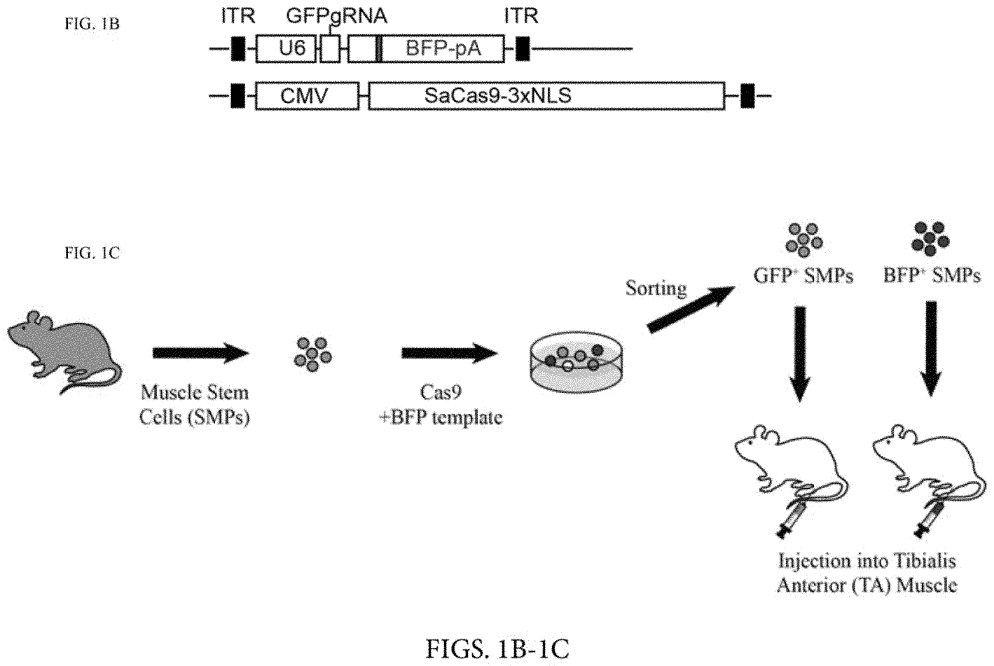

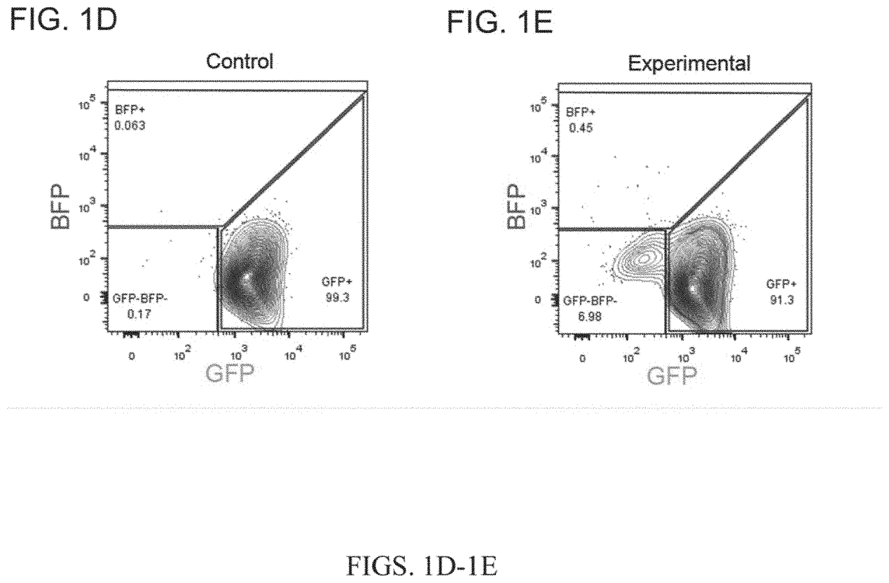

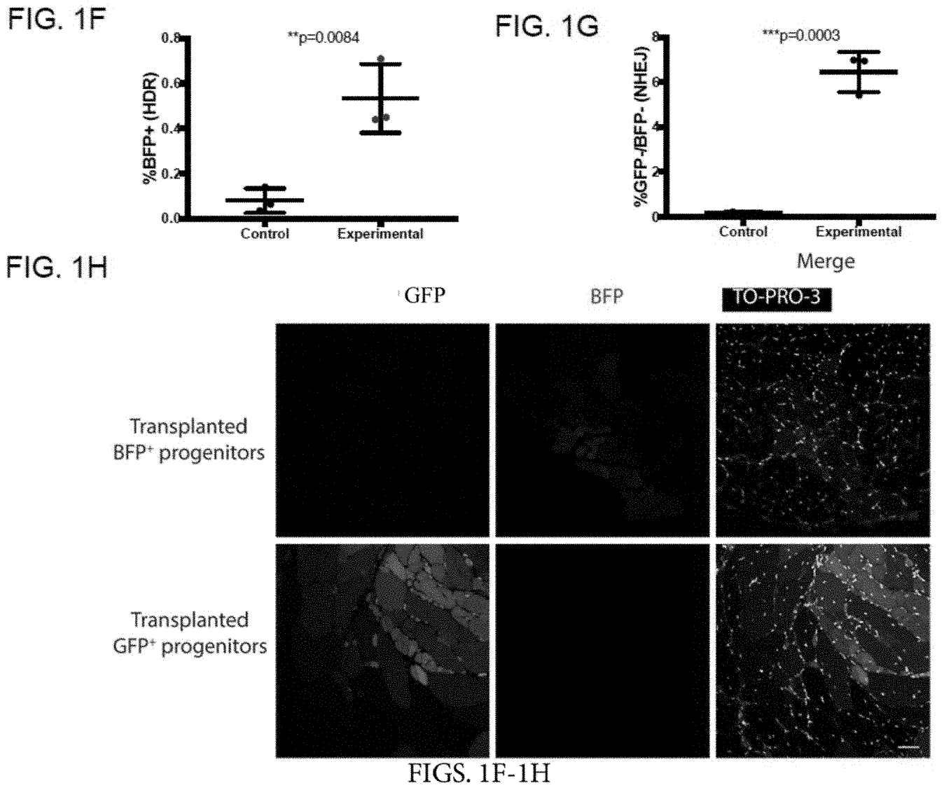

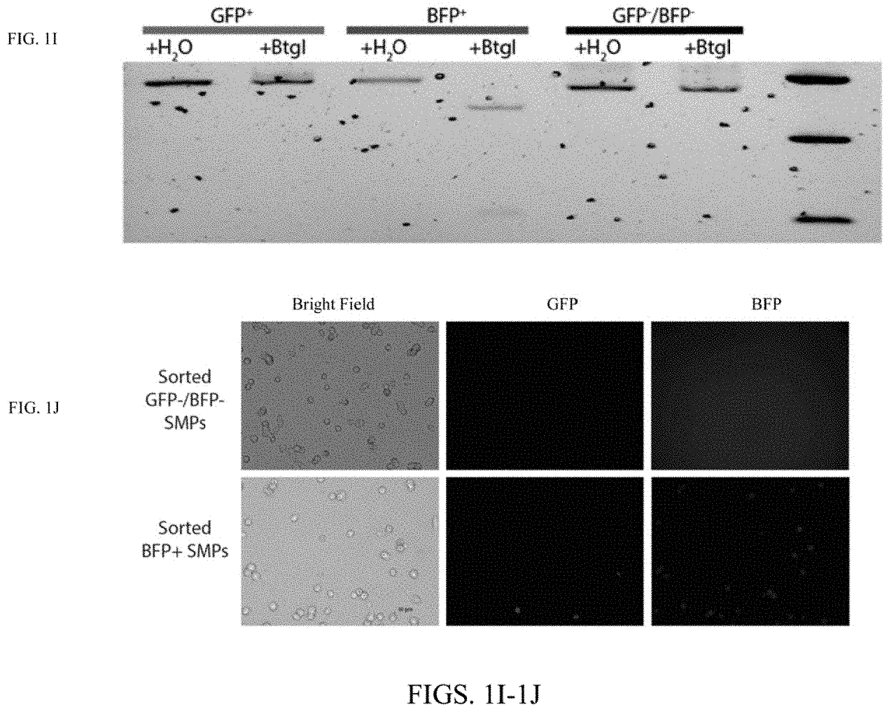

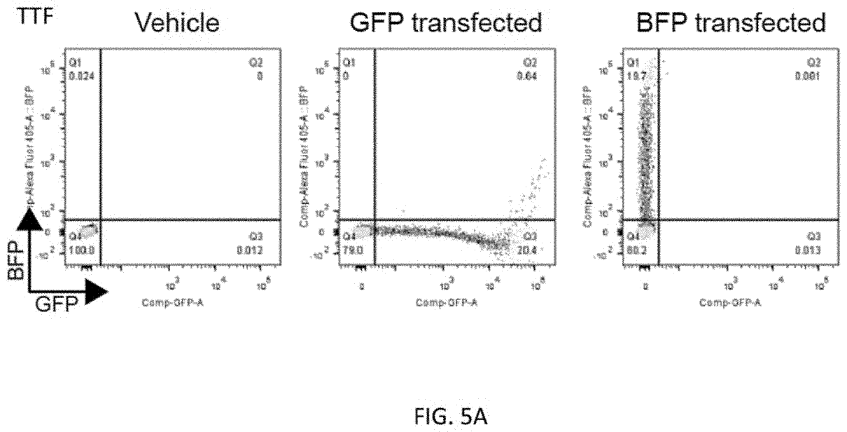

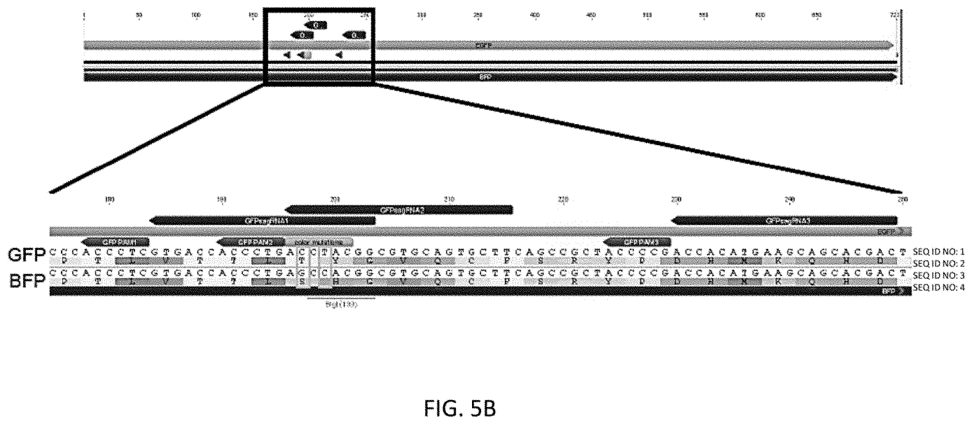

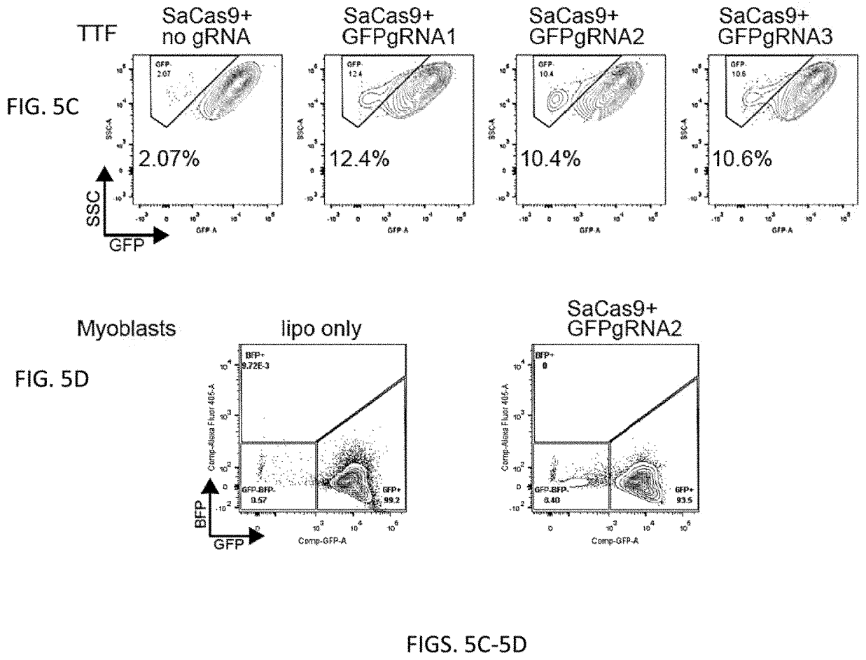

In order to sensitively detect in vivo gene editing events by CRISPR/Cas9, a fluorescent protein-based reporter system (FIG. 1A) using a transgenic mouse line that ubiquitously expresses strong enhanced green fluorescent protein (GFP) signal⁸ was developed. A blue fluorescent protein (BFP) sequence was designed based on published BFP variants⁹⁻¹¹ to carry a minimal 2-base substitution (C197G and T199C) compared to the GFP sequence. This simple modification allows for easy discrimination of the two fluorescent proteins by fluorescence-activated cell sorting (FACS) (FIGS. 5A-5D). The same 2-base substitutions also create a BtgI site for restriction fragment length polymorphism (RFLP) analysis. A single guide RNA (sgRNA) targeting the substitution sites in GFP was designed to be compatible with Cas9 protein from Staphylococcus aureus (SaCas9), and tested in tail tip fibroblasts (TTFs) from GFP^{+/-}; mdx mice for efficient disruption of GFP signal (FIGS. 5B, 5C). This gRNA was inserted into a vector with AAV backbone, together with a promoter-less BFP template lacking Kozak or start ATG sequences, for use in HDR experiments (FIG. 1B).

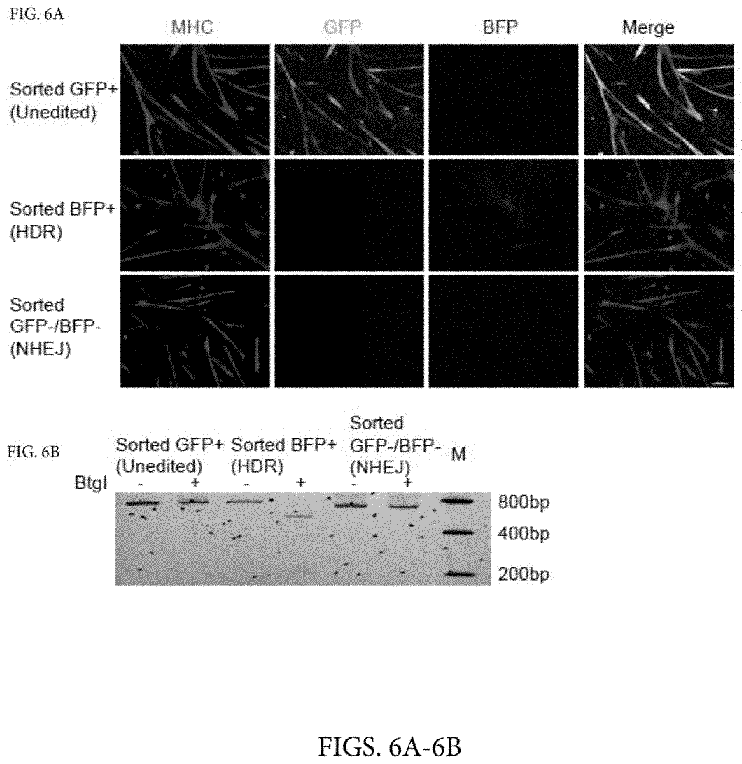

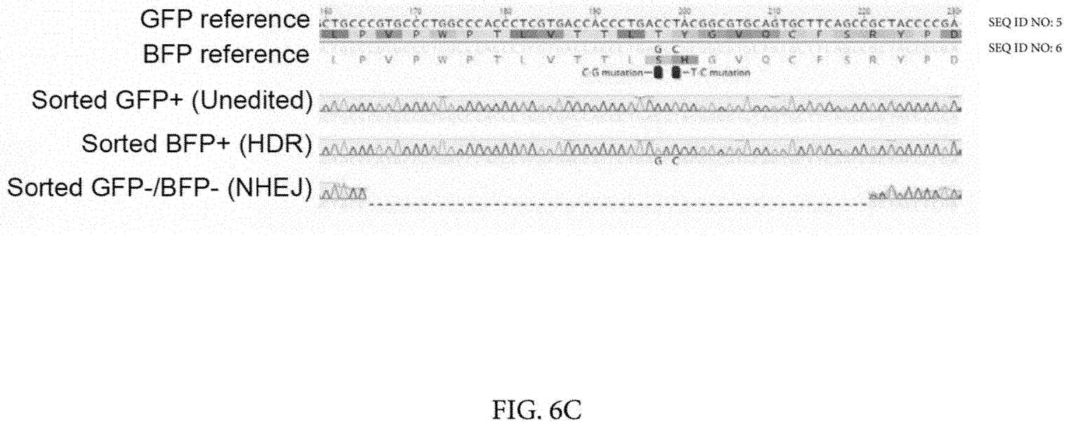

This colour-switch system was used to test the capacity of CRISPR/Cas9 to instigate HDR in a regenerative stem cell population. Satellite cells from skeletal muscle of GFP^{+/-}; mdx mice were isolated and, after ex vivo expansion^{12,13}, transfected with dual vectors consisting of AAV-SaCas9 and AAV-GFPgRNA-BFPtemplate (FIGS. 1B, 1C). This dual vector system was adopted as the ultimate aim was to deliver CRISPR/Cas9 and template in vivo and the limited cargo capacity of AAVs, ˜4.5-4.7 kb, prevented inclusion of all components in a single vector. Flow cytometry was then used to discriminate NHEJ and HDR events in the transfected cell population with single cell resolution. The experimental group, transfected with dual vectors, included cells exhibiting loss of green fluorescence (GFP-), indicative of imprecise NHEJ-mediated disruption of GFP reading frame, as well as cells exhibiting loss of GFP and gain of BFP signal (BFP+), indicative of HDR (FIGS. 1D-1G). In contrast, GFP−/BFP− and BFP+ cells were absent from control transfections, in which cells received AAV-GFPgRNA-BFPtemplate alone (FIGS. 1D-1G). Similarly, there was no gain of blue fluorescence observed when SaCas9 and gRNA were transfected without the BFP-template (FIG. 5D), indicating that NHEJ alone is unable to induce a GFP-to-BFP spectral shift. The GFP−/BFP− and BFP+ populations were separately sorted by FACS and validated by RFLP and Sanger sequencing, showing that they were edited by CRISPR-NHEJ and -HDR, respectively (FIGS. 1I, 1J, 6B, 6C). Finally, whether these ex vivo edited satellite cell-derived myoblasts retained muscle-forming capacity was investigated. Upon switching to differentiation media, CRISPR-NHEJ (GFP−/BFP−) and CRISPR-HDR (BFP+) myoblasts fused to form myosin heavy chain-positive myotubes (FIG. 6A). Furthermore, when transplanted into the pre-injured TA muscles of mdx mice, sorted BFP+ myoblasts contributed to in vivo muscle repair by giving rise to blue muscle fibres (FIG. 1H). These data indicate that the GFP/BFP colour-switching system developed here accurately and sensitively reports on genomic CRISPR-NHEJ and CRISPR-HDR editing events with single cell resolution and allows subsequent tracking of the in vivo regenerative output of the edited cells.

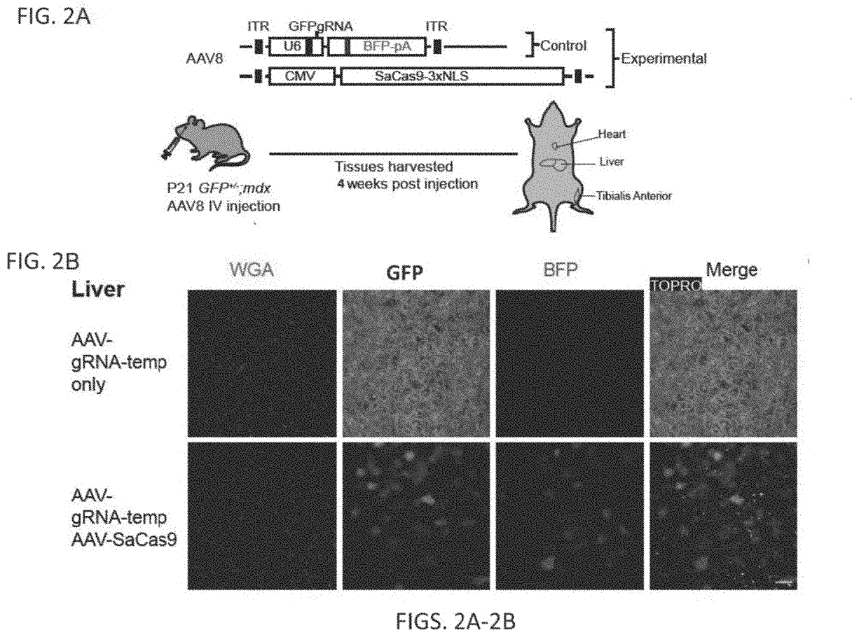

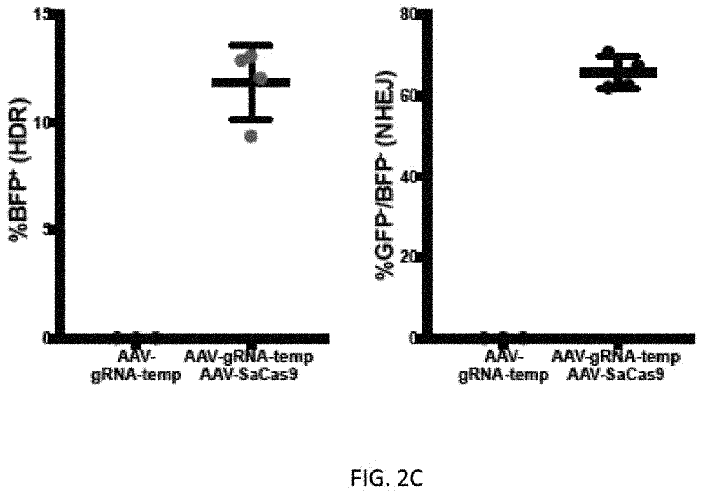

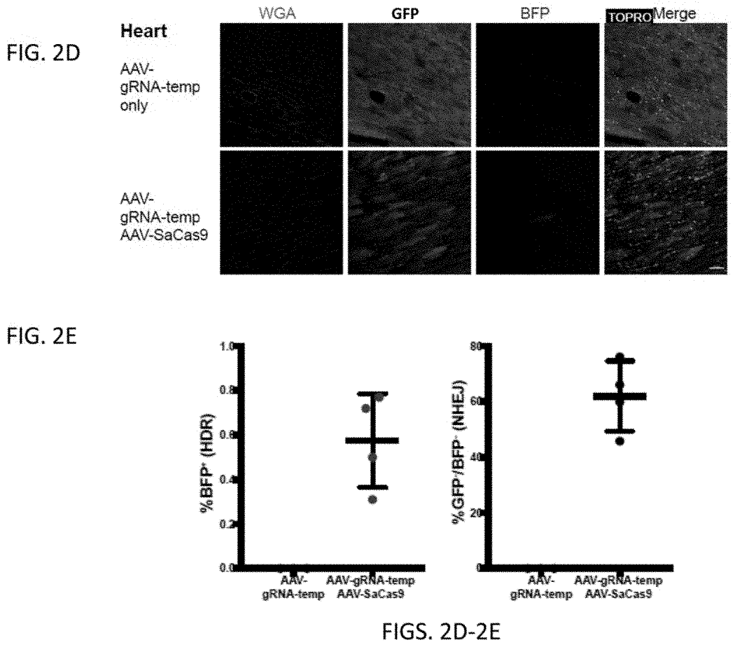

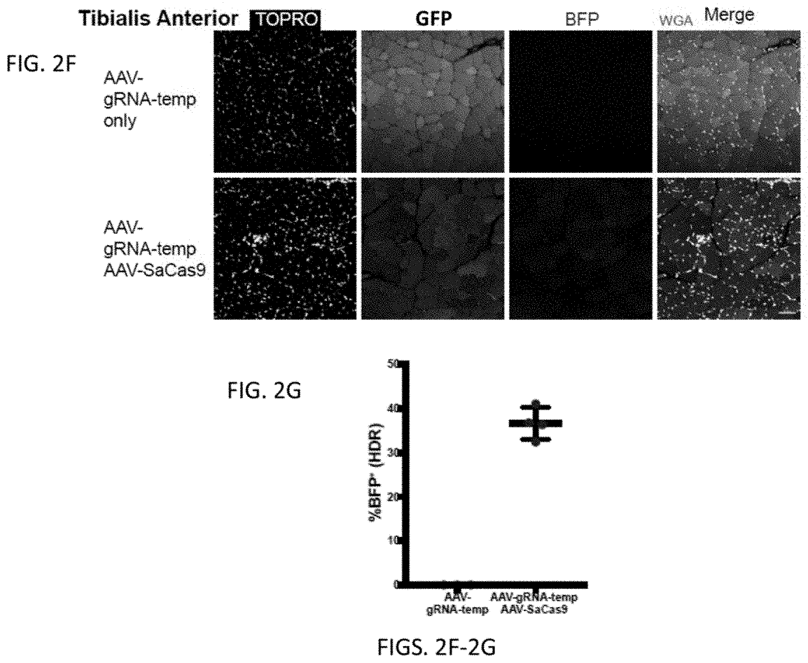

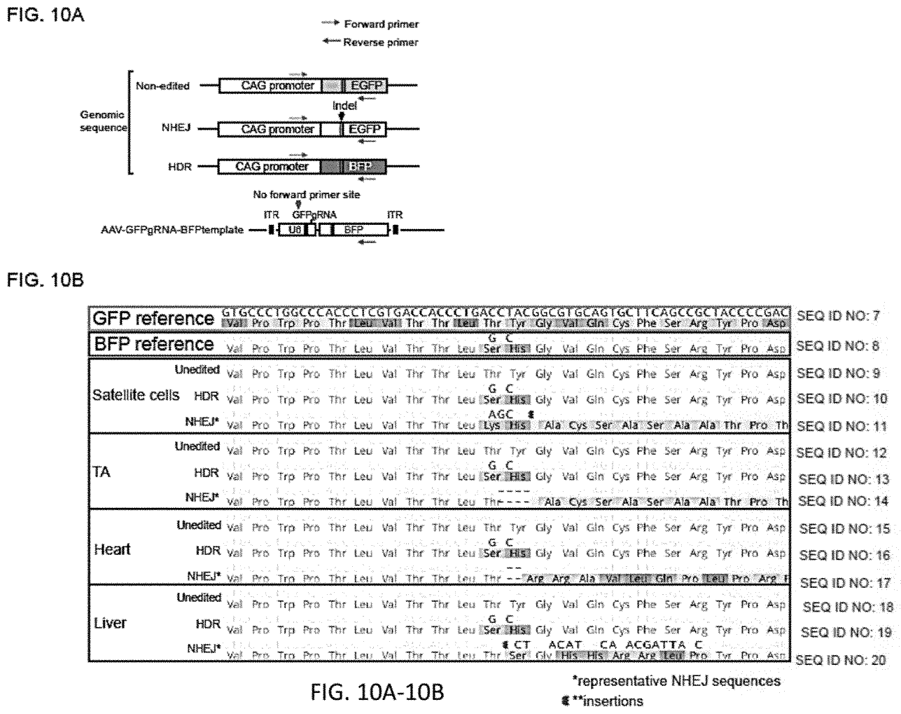

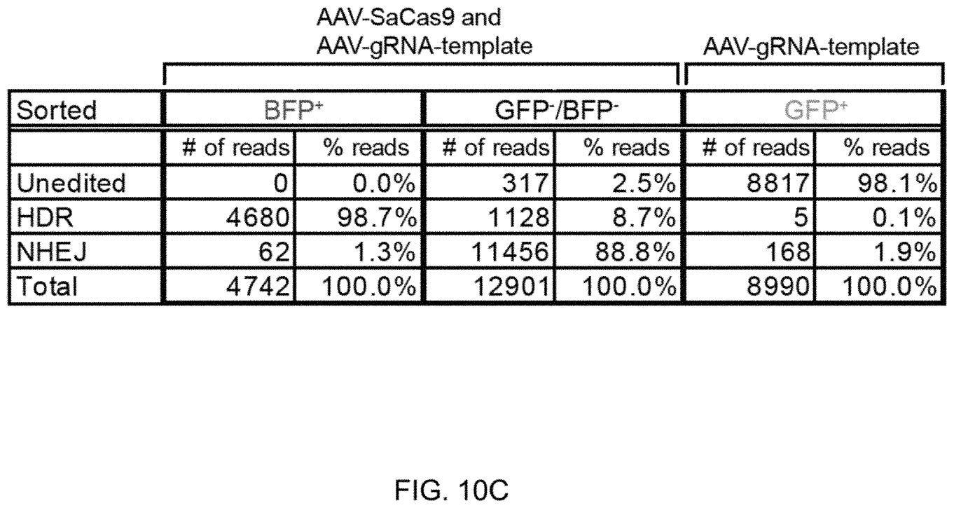

The utility of our reporter system for tracking CRISPR-mediated gene editing events in vivo was assessed. AAVs were generated using the aforementioned vectors and packaged with serotype 8, which has high liver, heart and skeletal muscle tropism¹⁴. CRISPR-HDR vectors were injected intravenously to juvenile (P21) male GFP^{+/-}; mdx mice (FIG. 2A). Control mice (AAV-control) received 1×10¹³ viral genomes (vg) per mouse of AAV-GFPgRNA-BFPtemplate alone, while experimental mice (AAV-HDR) received 1 x 10¹³ vg of AAV-GFPgRNA-BFPtemplate plus 5×10¹² vg of AAV-SaCas9. Mice were euthanized 3 weeks post-injection for analysis (FIG. 2A). Wide-spread loss of GFP signal and acquisition of BFP signal in the livers of all experimental mice injected with AAV-HDR, but not in AAV-control injected animals were detected (FIG. 2B). On average, 65.7% (range, 62-70%) of hepatocytes were NHEJ-edited and showed diminished GFP fluorescence, while 11.9% (range, 9-13%) of cells were HDR-edited to become BFP+ (FIG. 2C), consistent with recently reported CRISPR-HDR editing rates in the neonatal liver^{6,7}. The majority of BFP+ liver cells were also GFP-, adding confidence in the reporter system and quantification strategy. Next-generation sequencing further confirmed CRISPR-NHEJ and CRISPR-HDR editing in the livers of experimental mice (FIGS. 10A, 10B). While a recent paper¹⁵ raised concerns about liver toxicity in non-human primates and piglets systemically injected with high-dose AAVs (specifically, an AAV9 variant), no lethality or apparent adverse effects on overall health of AAV-injected mice was observed in this study. These studies confirm the sensitivity and accuracy of our fluorescence imaging-based system for quantifying CRISPR editing events in vivo without the need for immunostaining or signal amplification in sectioned tissues.

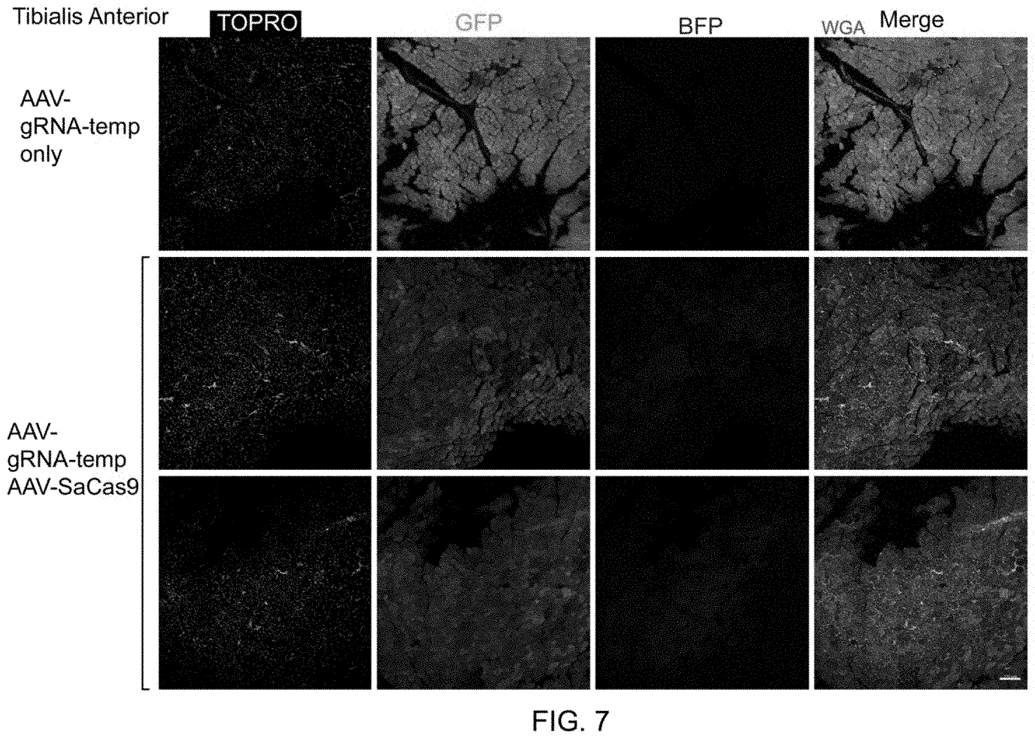

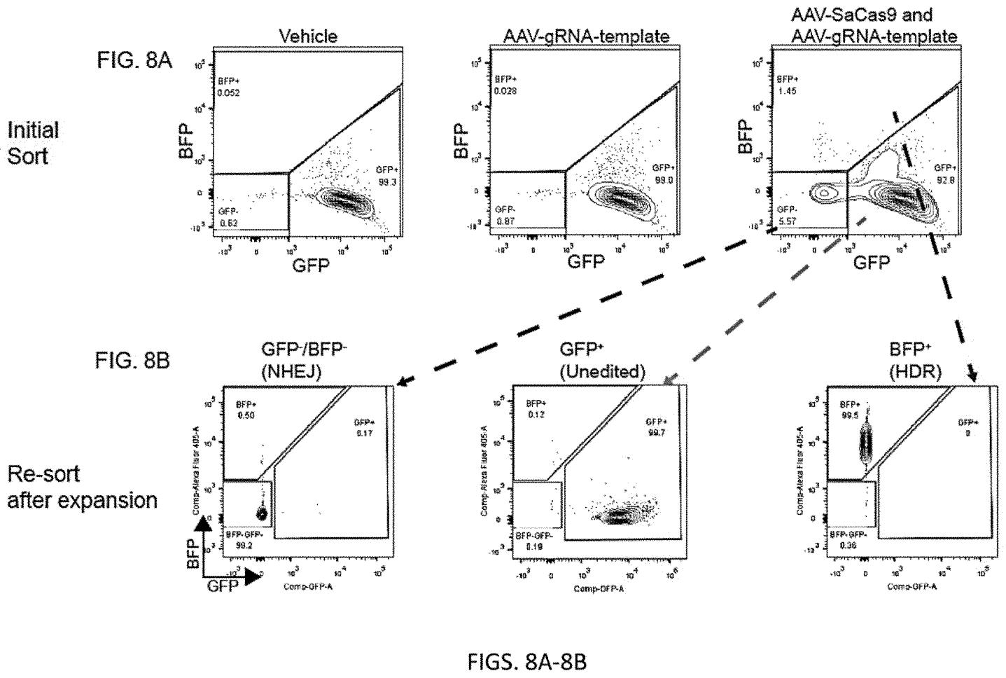

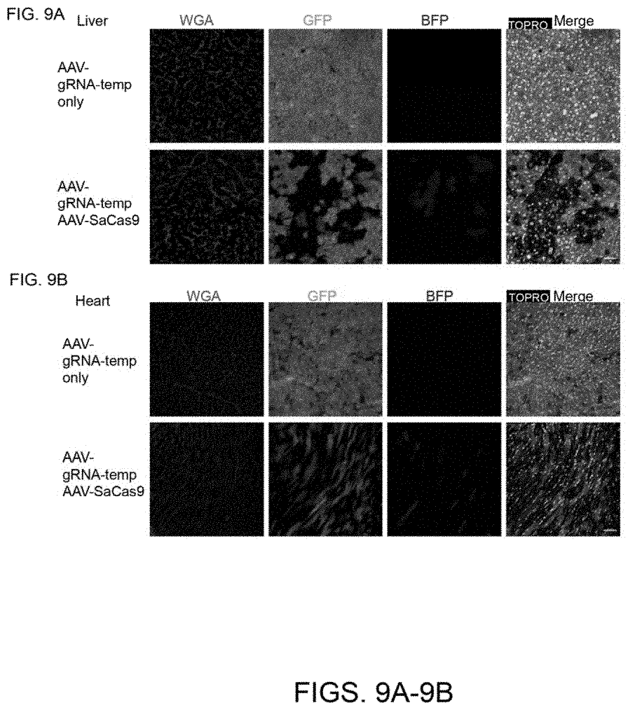

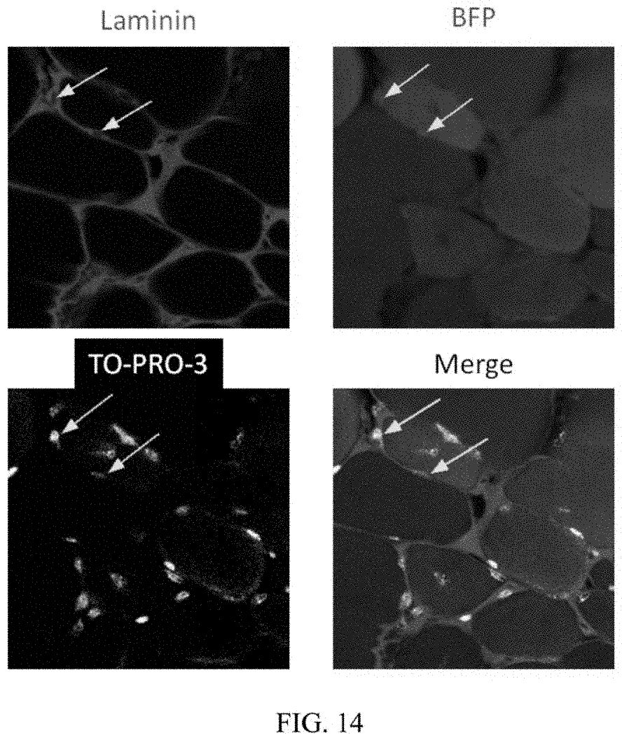

Skeletal muscle is a largely post-mitotic tissue, composed primarily of multinucleated muscle fibres formed by fusion of myogenic precursors derived from satellite cells. We and others have utilized AAV-CRISPR-mediated NHEJ in muscle to correct the Dmd reading frame and recover Dystrophin expression and function in dystrophic mdx mice by deleting or skipping Dmd exon 23¹⁶⁻¹⁸. However, prior attempts¹⁹ at AAV-CRISPR-mediated HDR in muscle produced negligible editing (only 0.18% alleles edited), possibly due to the use of a muscle-restricted promoter (CK8), which limits Cas9 expression to mature myofibres. We therefore evaluated possible CRISPR-HDR in skeletal muscle of mdx mice receiving systemic AAV-GFPgRNA-BFPtemplate plus AAV-SaCas9, controlled by broadly active regulatory elements that would express in muscle fibres and their precursors (FIG. 2A), in comparison to controls receiving AAV-GFPgRNA-BFPtemplate alone. Strikingly, we observed wide-spread BFP+ myofibres in the tibialis anterior (TA) muscles of all experimental mice (FIG. 2F, FIG. 7). In contrast, BFP+ fibres were absent in controls (FIG. 2F, FIG. 7). On average, 36.7% (range, 32-41%) of fibres were BFP+ in AAV-HDR injected mice (P21), indicating robust HDR-mediated gene replacement (FIG. 2G). While very few fibres showed complete loss of GFP signal (as expected since total loss of green fluorescence would require CRISPR/Cas9 targeting of all or nearly all of the hundreds of myonuclei in these cells), both HDR-edited and NHEJ-edited genomic sequences were detected and confirmed by NGS (FIGS. 9A-9B). Further, satellite cells in dual AAV treated mice were found to be BFP+ (FIG. 14).

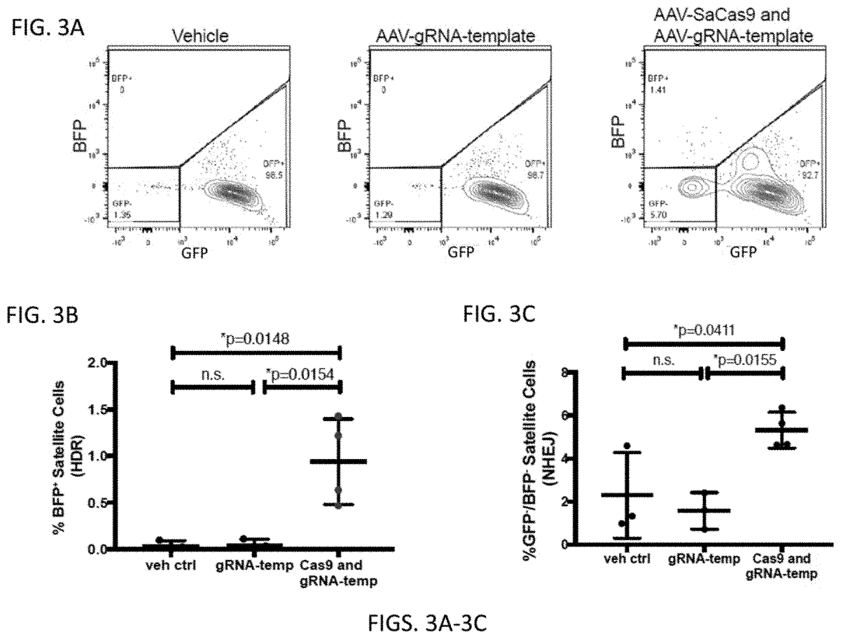

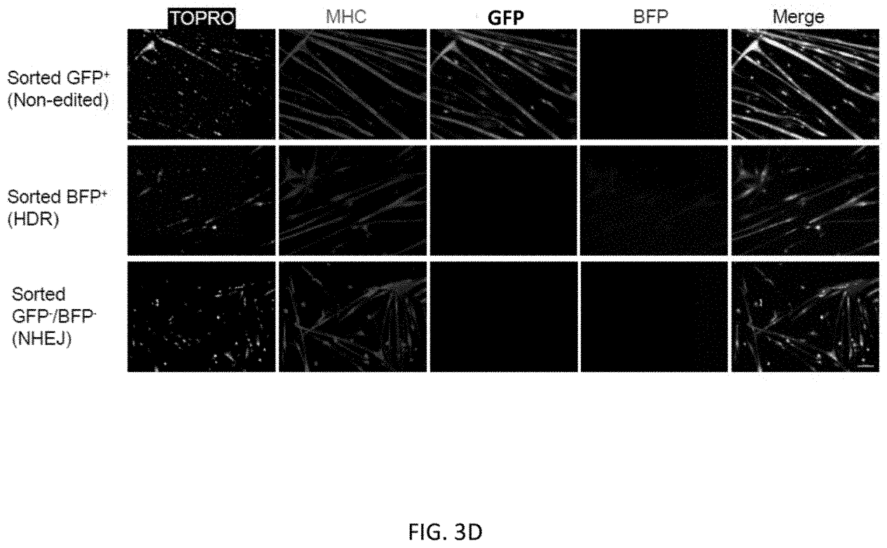

Given the relatively high percentages of BFP+ muscle fibres detected in this study, compared to the previously reported low efficiency of AAV-CRISPR-mediated HDR using a muscle fibre-restricted promoter¹⁹, we reasoned that skeletal muscle stem cells could have been targeted in our system, with subsequent incorporation of edited progenitors into myofibres. We therefore used an extensively validated surface marker profile (Ter119⁻CD45⁻Mac1⁻Sca1⁻CXCR4⁺ β1-integrin⁺) to isolate muscle stem cells from AAV-HDR injected mice^{12,13,20}. Consistent with previously published data from our group¹⁶, approximately 5% of FACS-isolated muscle stem cells were GFP−/BFP−, indicating in vivo disruption by AAV-CRISPR-NHEJ (FIGS. 3A, 3C). Importantly, we also detected a smaller population (−1%) of muscle stem cells that was BFP+, suggesting in vivo HDR-editing by AAV-CRISPR (FIGS. 3A, 3B). Gain of blue fluorescence and loss of green fluoresce in this population was validated by re-sorting of culture-expanded cells (FIG. 7) and sequencing analysis (FIG. 9). In order to test the myogenic function of these in vivo edited satellite cells, we expanded them in culture and performed ex vivo differentiation assays. In vivo NHEJ- and HDR-edited satellite cells retained the capacity to fuse to form GFP−/BFP− and BFP+ myotubes, respectively (FIG. 7D).

Similar to skeletal muscle, cardiac muscle is implicated in a wide-range of genetic diseases that could benefit from therapeutic gene editing in vivo; however, the postnatal heart exhibits limited proliferative activity and markedly poor regenerative capacity^{21,22}. We and others have documented AAV-CRISPR-mediated in vivo gene disruption in the heart in neonatal and juvenile mice, but the relative efficiencies of HDR versus NHEJ in this tissue have not been well studied^{16-19,23}. In the P21 systemic AAV-HDR injected mice, most cardiomyocytes (on average 62%) lost GFP signal, indicating high levels of NHEJ-mediated disruption of the genomic GFP sequence (FIGS. 2D-2E). BFP+cardiomyocytes were also present, though rare (˜0.58% on average), in all experimental mice (FIGS. 2D-2E). In contrast, neither GFP disruption nor BFP fluorescence were detected in AAV-control mice (FIGS. 2D-2E).

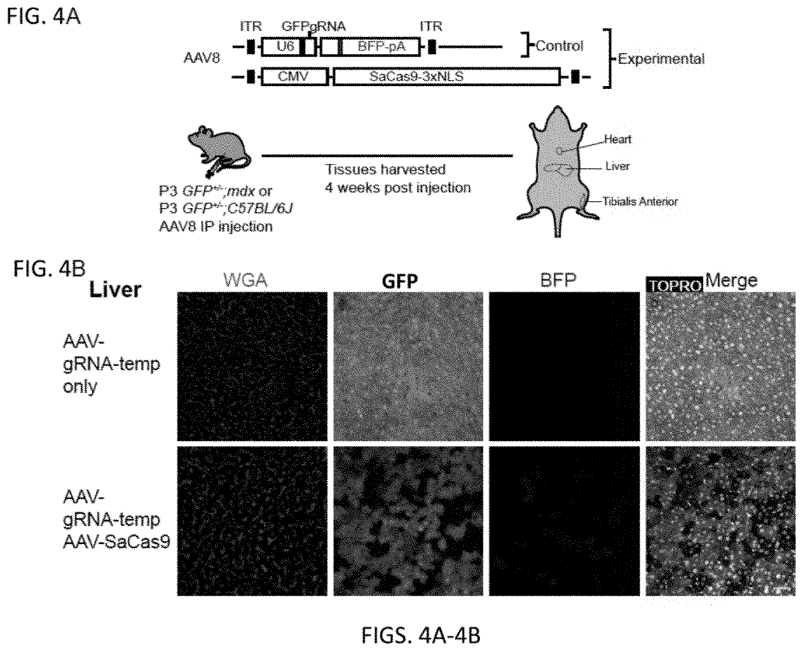

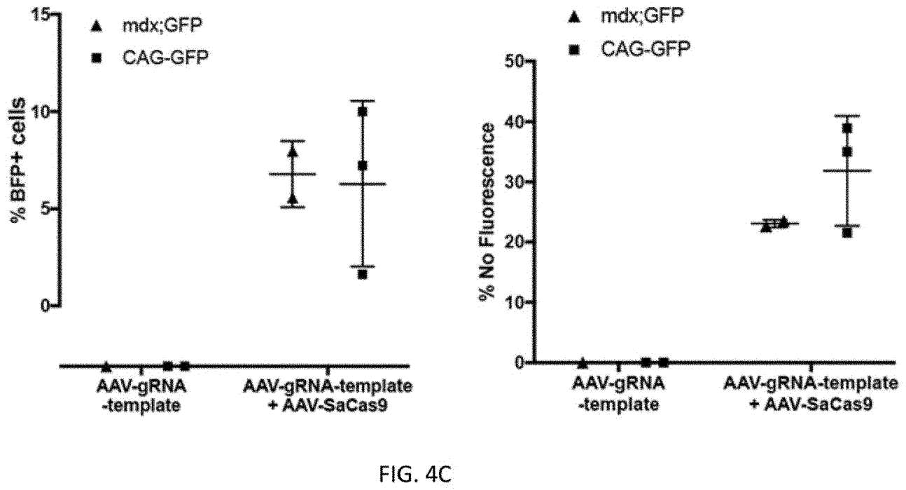

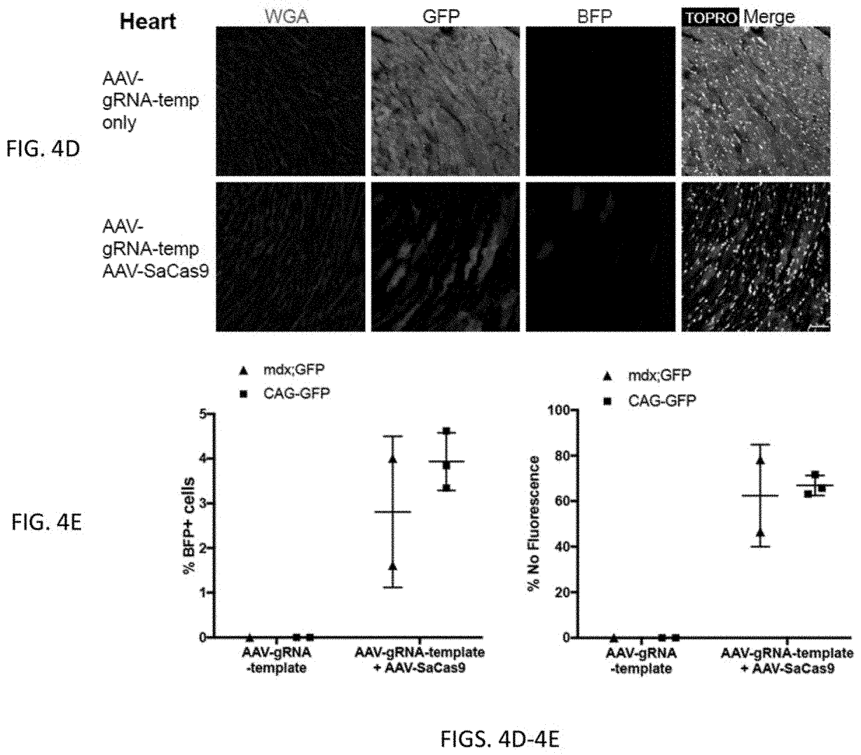

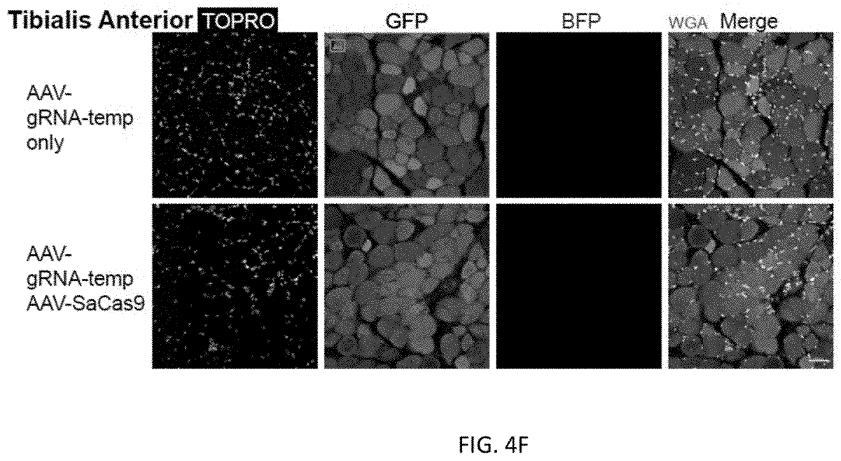

We hypothesized that the lack of proliferating cardiomyocytes after P21 in mice, and the negligible contributions from endogenous cardiac progenitor cells in homeostasis, might underlie the observed low rate of HDR in cardiac muscle, as opposed to skeletal muscle^{21,22,24.} We further reasoned that an earlier administration of AAVs could potentially increase HDR editing efficiencies in organs such as the heart that harbour proliferating cells neonatally but later become post-mitotic. We also wondered whether the mild pathophysiology of mdx mice²⁵ could affect cardiomyocyte editing efficiencies. Therefore, we administered AAV-HDR vectors to GFP^{+/-}; mdx or GFP^{+.-}; C57BL/6J (male and female) mice at P3, by intraperitoneal injection (FIG. 4A). AAV-control animals received 3×10¹² vg/mouse of AAV-gRNA-template alone and experimental mice (AAV-HDR) received the same dose of AAV-gRNA-template together with 1×10¹² vg/mouse AAV-SaCas9. Similar percentages of BFP+ and GFP−/BFP− liver cells were detected regardless of genetic background (FIG. 4B and FIG. 9A). In addition, the frequencies of BFP+ hepatocytes were comparable between the P3 and P21 experiments (average of ˜10% BFP+ hepatocytes; FIG. 2C and FIG. 4C). However, the frequency of NHEJ-edited liver cells was reduced in neonatally injected mice (on average, ˜28% of hepatocytes, FIG. 4C), possibly reflecting the more vigorous proliferation rate of early neonatal hepatocytes, which may lead to more rapid dilutional loss of the non-integrating AAV episomes²⁶.

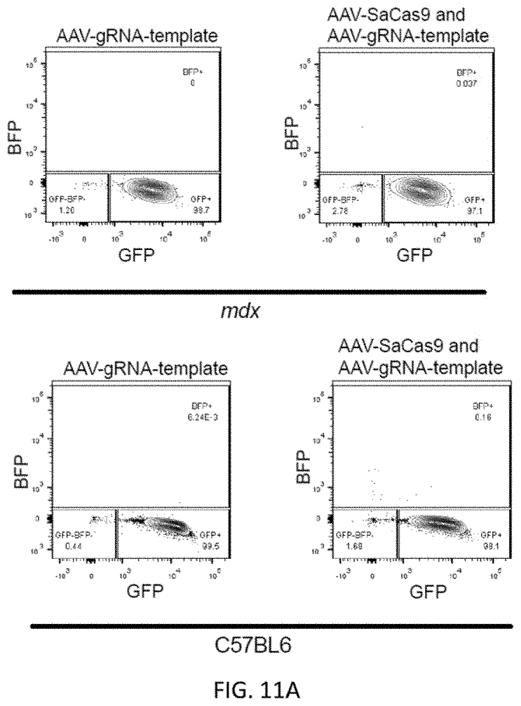

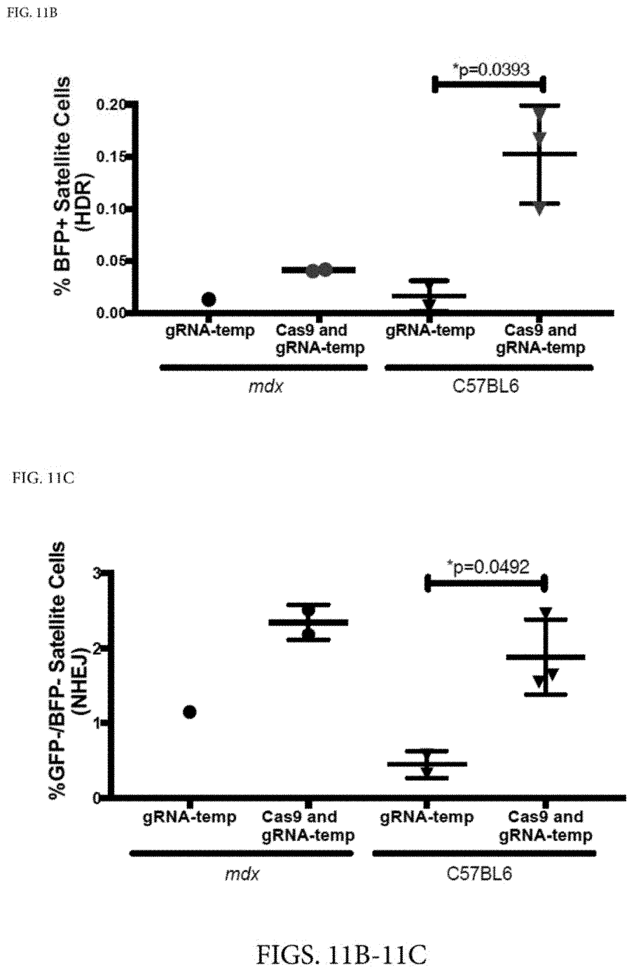

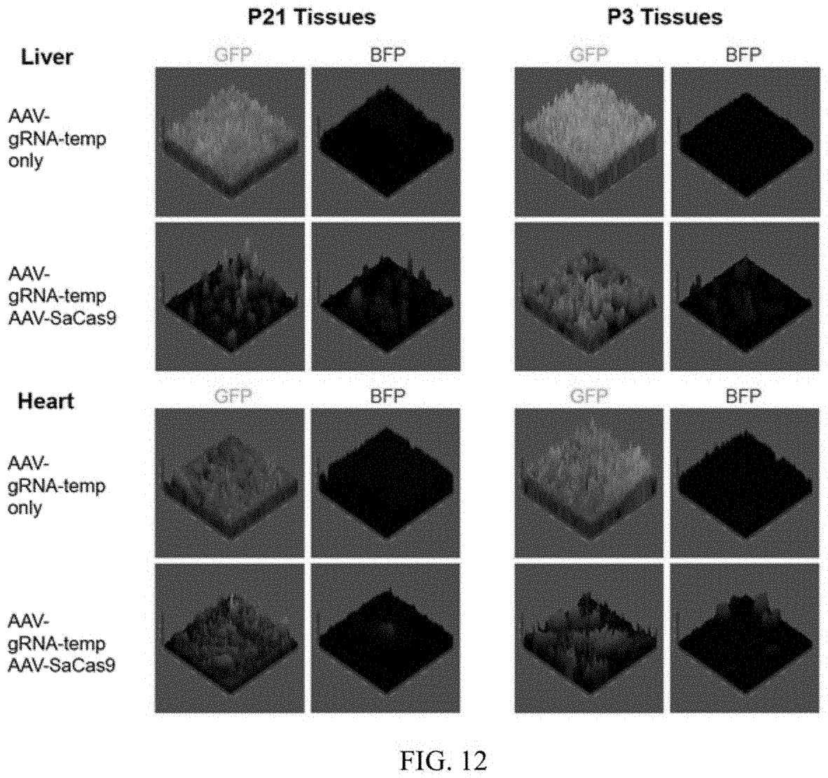

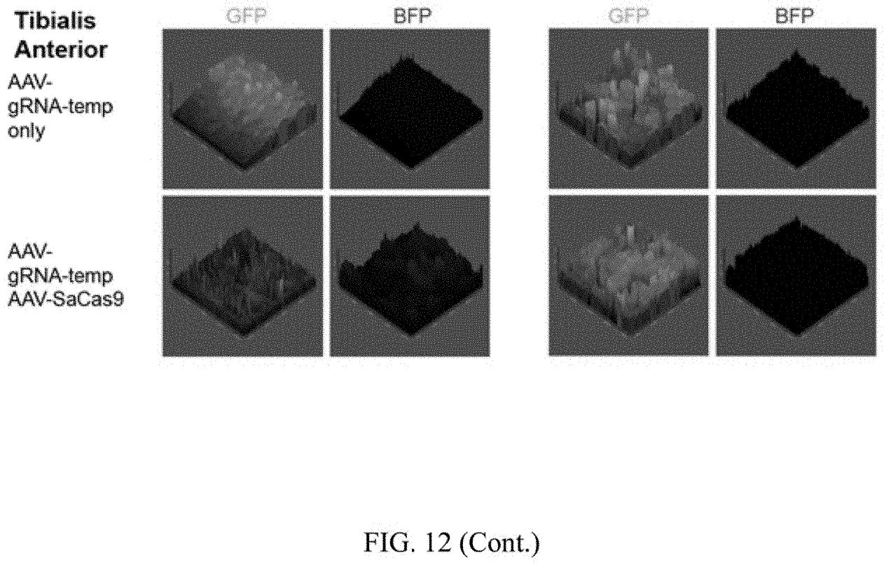

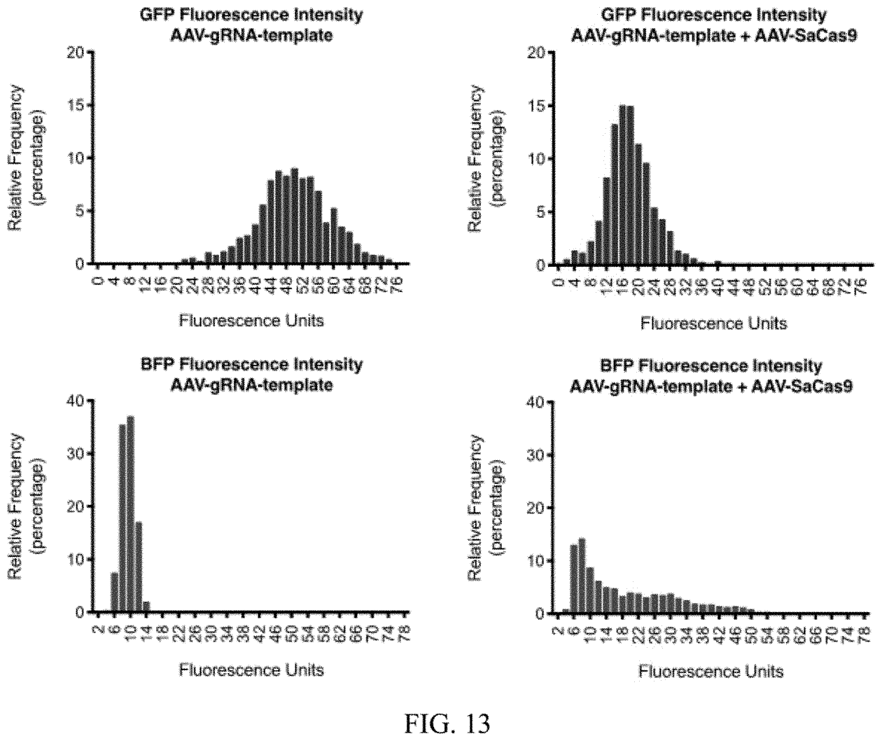

We also evaluated HDR rates in cardiac and skeletal muscles after systemic administration of AAV-CRISPR in P3 neonates. BFP+ cells accounted for an average 3.5% (range, 1.6%-4.6%) of cardiomyocytes, a frequency significantly greater than the frequency of BFP+ cells in the hearts of P21 injected mice (FIGS. 4D-4E and FIGS. 2D-2E). GFP−/BFP− cardiomyocytes were detected at a similar rate (>60%) between the two experiments (FIG. 2E and FIG. 4E), suggesting that the observed age-dependent differences in HDR are unlikely to reflect differences in the efficiency of AAV transduction. In contrast, we did not observe substantial gain of BFP signal in skeletal muscle sections in either mdx or C57BL/6J backgrounds (FIG. 4F), consistent with the infrequent BFP+ skeletal muscle satellite cells in these muscles (0.05-0.17% BFP+, FIG. 11). Loss of GFP signal could not be evaluated due to the confounding influence of myofibre multi-nucleation, as discussed above. Together, these data reveal discrete, developmentally timed restrictions on in vivo CRISPR-HDR gene editing in striated muscle, offering the possibility to target (or de-target) specific tissues of interest by adjusting the timing of AAV-CRISPR administration. Whether similar developmentally controlled windows of CRISPR-HDR accessibility exist for other cell types will be an intriguing avenue for future investigation.

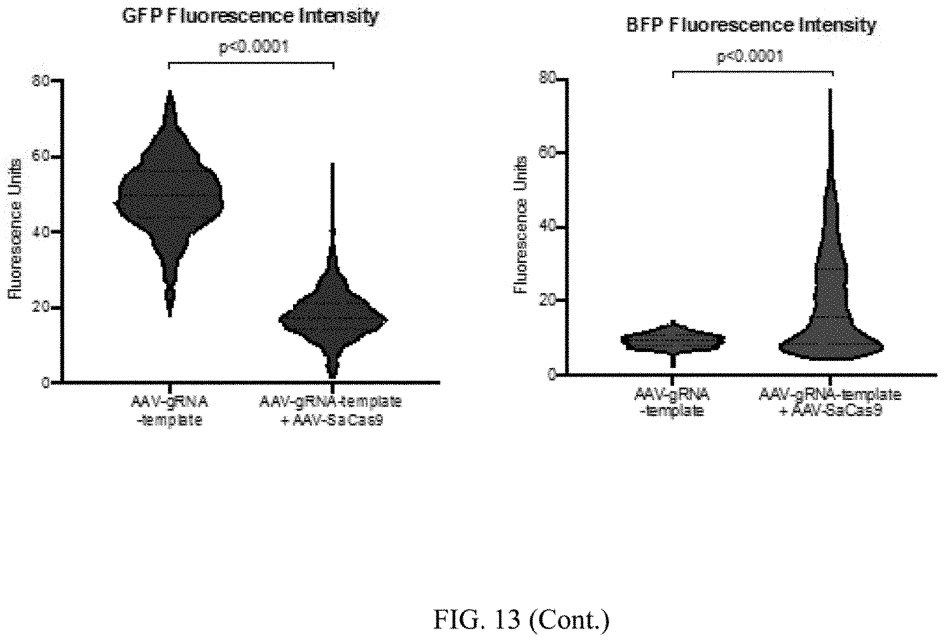

The results discussed above are further validated by the data shown in FIG. 13, showing a loss of GFP in liver, heart, and muscle (TA) of dual AAV treated animals (both P3 and P21), as well as a preferential gain in BFP signal (indicating HDR) in P21 muscle tissue and P3 heart tissue.

The inventors have surprisingly and unexpectedly found that postnatal cardiac muscle, skeletal muscle, and muscle stem cells undergo templated HDR at different developmental time points in mice, using a GFP-BFP colour-switching reporter system that enables in vivo tracking of genome-editing outcomes at the single cell level. Systemic delivery of CRISPR-Cas9 editing components via adeno-associated virus (AAV-CRISPR) confirmed efficient NHEJ and HDR in liver, consistent with previous reports (Yang, Y. et al. Nat Biotechnol 34, 334-338 (2016); Yin, H. et al. Nat Biotechnol 34, 328-333 (2016)). In addition, HDR-edited muscle stem cells and myofibres were detected in mice injected with AAV-CRISPR at post-natal day 21 (P21), but not at P3, while HDR-edited cardiac cells were detected in P3-injected, but rarely in P21-injected, animals. Our results reveal the possibility of sequence-directed, systemically disseminated, in vivo AAV-CRISPR-mediated HDR in striated muscle and muscle stem cells at discrete postnatal time points, providing new opportunities for therapeutics development.

In conclusion, our study reports a simple yet powerful tool to track NHEJ and HDR gene editing outcomes in vivo with single cell resolution. Furthermore, by systemic delivery of gRNA-programmed Cas9 via AAV, we reveal an unexpected opportunity for precise, targeted gene replacement by HDR in skeletal and cardiac muscles, both largely post-mitotic tissues that have been widely considered to be inaccessible by this approach. To our knowledge, our data provide the first demonstration of significant in vivo HDR-editing in the postnatal heart via systemic AAV delivery of CRISPR/Cas9, and represent a substantial improvement over previously reported HDR editing rates achievable in skeletal muscle via local, intramuscular delivery^{9,27}. Our study also provides the first demonstration of successful HDR-editing in tissue stem cells within their native niche, which will uniquely enable directed manipulation of stem cell genomes therapeutically and experimentally, without the need to isolate, expand or transplant these rare cells. Ultimately, the ability to inscribe irreversible and potentially enduring precise genome modification in the neonatal mammalian heart and postnatal mammalian skeletal muscle satellite cells opens exciting new avenues for future therapeutic interventions for many currently intractable cardiac and muscle diseases.

Animals

Hemizygous GFP transgenic mice, carrying a single trangenic allele, were generated by crossing CAG-GFP mice⁸ with either C57BL/6J or C57BL/10ScSn-Dmd^{mdx}/J (mdx) (Jackson Labs). Postnatal day 3 (P3) GFP^{+/-}; mdx and GFP^{+/-}; C57BL/6J pups (both male and female) were used for neonatal intraperitoneal (IP) injections and 3 week old male GFP^{+/-}; mdx mice were used for juvenile intravenous (retro-orbital) injections. Mice were maintained at the Harvard Biological Research Infrastructure according to animal care and experimental protocols approved by the Harvard University Institutional Animal Care and Use Committee (IACUC).

AAV Production and Administration

AAVs were produced and titered by the Gene Transfer Vector Core (GTVC) at the Grousbeck Gene Therapy Center at the Schepens Eye Research Institute and Massachusetts Eye and Ear Infirmary (SERI/MEEI), packaged with serotype 8 as previously described²⁸. Briefly, semi-confluent HEK293 cells were transfected with rep2-cap8 packaging construct, an adenoviral helper function plasmid, and the ITR flanked transgene construct. Three days following transfection, media and cells were harvested, underwent lysis and benzonase digestion for removal of non-particle associated DNA. Particles were purified and concentrated using tangential flow filtration, iodixanol density centrifugation, and buffer exchange in to a PBS-based buffer solution. For neonatal (P3) intraperitoneal injections, control mice received 3×10¹² viral genome (vg) of AAV-GFPgRNA-BFPtemplate alone, and experimental mice received 3×10¹² vg of AAV-GFPgRNA-BFPtemplate plus 1×10¹² vg of AAV-SaCas9. Virus was diluted in 75 μL of vehicle (PBS with 35 mM NaCl) for each injection. Mice were euthanized for analysis 4 weeks post injection. For juvenile (P21) retro-orbital injections, control mice received 1×10¹³ vg of AAV-GFPgRNA-BFPtemplate alone, and experimental mice received 1×10¹³ vg of AAV-GFPgRNA-BFPtemplate plus 5×10¹² vg of AAV-SaCas9. Virus was diluted in 312 μL of vehicle (PBS with 35 mM NaCl) for each injection. Mice were euthanized for analysis 3 weeks post injection.

Gene Editing Constructs

The AAV-SaCas9 plasmid was previously described¹⁶. AAV-GFPgRNA-BFPtemplate plasmid was generated by Gibson assembly of the pZac2.1 AAV vector with three inserts. The vector was double digested by HindIII-HF and NotI-HF (NEB). Insert piece 1 (U6-GFPgRNA) was PCR amplified from a plasmid containing U6-GFPgRNA. Insert 2 (BFP) was PCR amplified from a BFP sequence synthesized as a gBlock (IDT). Insert 3 (polyA) was PCR amplified from genomic DNA of the CAG-GFP transgenic animal. Two base substitutions on the BFP template enable the color switch (from green to blue fluorescence) and generate a restriction fragment length polymorphism (RFLP) detectable by BtgI restriction enzyme.

Satellite Cell Isolation, Culture and Differentiation

Satellite cells for ex vivo gene editing were isolated as previously described¹². For isolation of in vivo edited satellite cells, triceps, abdominal and hind limb muscles from half of the body were harvested and minced using scissors, then subjected to two rounds of digestion with 0.2% Collagenase type II and 0.05% Dispase in DMEM (GIBCO) at 37° C. (for 15 min, then 10 min). Enzymes were inactivated by addition of FBS, and cells were centrifuged and filtered through 70 um strainers before staining for 30 min with an antibody cocktail containing APC-Cy7-CD45 (Biolegend, 1:200), APC-Cy7-CD11b (Biolegend, 1:200), APC-Cy7-TER119 (Biolegend, 1:200), APC-Scal (Biolegend, 1:200), PE-CD29 (Biolegend, 1:100) and Biotin-CD184 (BD Biosciences, 1:100). After primary antibody incubation, cells were washed with staining media (SM, Hank's Balanced Salt Solution+2% serum) and then stained for an additional 20 min with Streptavidin PE-Cy7 (Biolegend, 1:200). Finally, cells were washed twice in SM, and resuspended in SM containing propidium iodide (PI) to mark dead cells. Satellite cells were sorted using a FACS Aria II (BD Biosciences) based on their lack of PI incorporation and CD45, Ter119, Scal and CD11b expression and positive expression of CXCR4 (CD184) and β1-integrin (CD29), a surface marker profile that has been extensively validated in multiple publications^{12,13,20} to select Pax7+ cells with robust myogenic capacity. Separately sorted GFP+, BFP+, and GFP−/BFP− satellite cells were expanded on collagen type I (1 ug/mL, Sigma) and laminin (10 ug/mL, Invitrogen) coated plates in Growth Media (F10, 20% horse serum, 1% Pen Strep, and 1% Glutamax (Gibco)), supplemented daily with 5 ng/mL bFGF (Sigma). DNA was isolated from subset of the expanded cells was harvested using QuickExtract (Lucigen) and used for genomic PCR and subsequent RFLP and sequencing analysis. Myogenic differentiation was initiated by switching to Differentiation Media (DMEM, 2% horse serum, 1% Pen Strep, 1% Glutamax (Gibco)) for 3-4 days. Cells were fixed by 4% PFA for 20 minutes for imaging.

Transfection

Satellite cells isolated from male mdx; GFP^{+/-}animals were expanded in culture in Growth Media with daily bFGF supplementation for 2-3 weeks and then re-plated onto 24 well plates coated with collagen (1 ug/mL) and laminin (10 ug/mL) at 20,000 cells per well. Myoblasts were transfected on day 2 using Lipofectamine 3000 (Invitrogen) per manufacturer's instructions with AAV-GFPgRNA-BFPtemp plasmid alone for control group or AAV-GFPgRNA-BFPtemp and AAV-SaCas9 plasmids at 5:1 ratio for experimental group (3 independent transfections per group). BFP⁺ and GFP⁺ cells were sorted using a FACS Aria II 5 days after transfection and resorted after an additional 2 weeks expansion in vitro to confirm fluorescence. Re-sorted cells were then used for in vitro differentiation and in vivo transplantation assays.

For testing GFP disruption in mdx; GFP^{+/-}primary myoblasts, cells were transfected with Lipofectamine only (control) or with plasmids encoding SaCas9 and GFPgRNA2 (no BFP template) at 1:1 ratio, as described above.

For screening of GFP-targeting gRNAs, mdx; GFP^{+/-} tail tip fibroblasts (TTFs) were transfected with SaCas9 alone (control) or with SaCas9 plus one of the three gRNAs targeting GFP, using Lipofectamine 3000 per manufacturer's instructions.

Myoblast Transplantation

One day before myoblast transplantation, 25 μL of Naja mossambica mossambica cardiotoxin (0.03 mg/mL, Sigma) was injected to the tibialis anterior (TA) muscles of anesthetized male mdx recipient mice. 800,000 GFP⁺, 800,000 BFP⁺ myoblasts or vehicle (PBS) alone was injected into the pre-injured TA muscles (N=4 TA muscles). The injected TA muscles were harvested 5 weeks post-transplantation for cryosectioning and fluorescence detection.

Genomic PCR and RFLP Analysis

Genomic DNA from tissues, satellite cells and expanded myoblasts was extracted using QuickExtract DNA Extraction Solution (Epicentre/Lucigen) per manufacture protocol. 1-2 μL of QuickExtracted solutions was used per 25 μL PCR reaction by Q5 Hot Start polymerase (NEB). Forward primer GTGCTGTCTCATCATTTTGGC (SEQ ID NO: 21) (binds upstream of GFP/BFP start site) and Reverse primer TCGTGCTGCTTCATGTGGTC (SEQ ID NO: 22) (binds downstream of Cas9 cutting site and color switching substitutions) were used to amplify the genomic transgene locus, but not template sequence. For RFLP analysis, PCR products were purified using QIAquick PCR Purification Kit (Qiagen) and digested with BtgI (NEB), or mock digested with water, before gel electrophoresis on E-Gel EX 2% Agarose Gels (Invitrogen).

Sanger Sequencing and Next-Generation Sequencing

Purified genomic PCR products were cloned into TOPO backbone using Zero Blunt TOPO PCR cloning kit (Invitrogen) and transformed into TOP10 competent cells (Invitrogen). Discrete clones were analyzed by bacterial colony Sanger sequencing, performed by Genewiz in Cambridge, Mass. Sequencing traces were aligned to the GFP transgene using Geneious program. For next-generation sequencing, 8 base pair (bp) barcodes were appended to the genomic PCR primers. 4-10 uniquely barcoded PCR products were pooled, and PCR purified before analysis at the MGH DNA core by CRISPR Sequencing (available on the worldwide web at //dnacore.mgh.harvard.edu/). NGS results were analyzed using CRISPResso program after demultiplexing. Representative NGS sequences are shown.

Sectioning and Fluorescent Imaging

Tissues were dissected and immediately fixed in 4% PFA for 90 min. at room temperature and then washed with PBS and transferred to 30% sucrose for overnight incubation at 4° C. Submersed tissues were then embedded in O.C.T. compound (Tissue-Tek) and frozen in isopentane in a liquid nitrogen bath. Tissues were sectioned using Microm HM550 (Thermo Scientific) and stained with Alexa Fluor 555-Wheat Germ Agglutinin and TO-PRO-3 Iodide (Life Technologies) according to manufacturer's instructions. Numbers of BFP⁺, GFP⁻ (also BFP⁻) and total cells were quantified manually by ImageJ. For liver and heart, three representative fields with ˜200-350 cells per field were counted for each tissue. For P21-injected TA sections, images of stitched fields (25 of 20× images) were counted with more than 1000 cells per image.

Statistical Analysis

GraphPad Prism 7.0 software was used for performing statistical analysis. Unpaired two-tailed t test was performed for FIGS. 1F-1G. One-way ANOVA with Tukey's multiple comparisons test was performed for FIGS. 3B-3C and FIGS. 11B-11C. Exact p values and degrees of freedom (DF) can be found in corresponding figure legends.

### REFERENCES

1. Symington, L. S. & Gautier, J. Double-strand break end resection and repair pathway choice. Annu Rev Genet 45, 247-271, doi:10.1146/annurev-genet-110410-132435 (2011).

2. Heyer, W. D., Ehmsen, K. T. & Liu, J. Regulation of homologous recombination in eukaryotes. Annu Rev Genet 44, 113-139, doi:10.1146/annurev-genet-051710-150955 (2010).

3. Ishizu, T. et al. Targeted Genome Replacement via Homology-directed Repair in Non-dividing Cardiomyocytes. Sci Rep 7, 9363, doi:10.1038/s41598-017-09716-x (2017).

4. Long, C. et al. Prevention of muscular dystrophy in mice by CRISPR/Cas9-mediated editing of germline DNA. Science 345, 1184-1188, doi:10.1126/science.1254445 (2014).

5. Nishiyama, J., Mikuni, T. & Yasuda, R. Virus-Mediated Genome Editing via Homology-Directed Repair in Mitotic and Postmitotic Cells in Mammalian Brain. Neuron 96, 755-768 e755, doi:10.1016/j.neuron.2017.10.004 (2017).

6. Yang, Y. et al. A dual AAV system enables the Cas9-mediated correction of a metabolic liver disease in newborn mice. Nat Biotechnol 34, 334-338, doi:10.1038/nbt.3469 (2016).

7. Yin, H. et al. Therapeutic genome editing by combined viral and non-viral delivery of CRISPR system components in vivo. Nat Biotechnol 34, 328-333, doi:10.1038/nbt.3471 (2016).

8. Wright, D. E. et al. Cyclophosphamide/granulocyte colony-stimulating factor causes selective mobilization of bone marrow hematopoietic stem cells into the blood after M phase of the cell cycle. Blood 97, 2278-2285 (2001).

9. Chu, V. T. et al. Increasing the efficiency of homology-directed repair for CRISPR-Cas9-induced precise gene editing in mammalian cells. Nat Biotechnol 33, 543-548, doi:10.1038/nbt.3198 (2015).

10. Glaser, A., McColl, B. & Vadolas, J. GFP to BFP Conversion: A Versatile Assay for the Quantification of CRISPR/Cas9-mediated Genome Editing. Molecular therapy. Nucleic acids 5, e334, doi:10.1038/mtna.2016.48 (2016).

11. Richardson, C. D., Ray, G. J., DeWitt, M. A., Curie, G. L. & Corn, J. E. Enhancing homology-directed genome editing by catalytically active and inactive CRISPR-Cas9 using asymmetric donor DNA. Nat Biotechnol 34, 339-344, doi:10.1038/nbt.3481 (2016).

12. Cerletti, M. et al. Highly efficient, functional engraftment of skeletal muscle stem cells in dystrophic muscles. Cell 134, 37-47 (2008).

13. Sherwood, R. I. et al. Isolation of adult mouse myogenic progenitors: functional heterogeneity of cells within and engrafting skeletal muscle. Cell 119, 543-554, doi:10.1016/j.cell.2004.10.021 (2004).

14. Zincarelli, C., Soltys, S., Rengo, G. & Rabinowitz, J. E. Analysis of AAV serotypes 1-9 mediated gene expression and tropism in mice after systemic injection. Mol Ther 16, 1073-1080, doi:10.1038/mt.2008.76 (2008).

15. Hinderer, C. et al. Severe Toxicity in Nonhuman Primates and Piglets Following High-Dose Intravenous Administration of an Adeno-Associated Virus Vector Expressing Human SMN. Human gene therapy, doi:10.1089/hum.2018.015 (2018).

16. Tabebordbar, M. et al. In vivo gene editing in dystrophic mouse muscle and muscle stem cells. Science 351, 407-411, doi:10.1126/science.aad5177 (2016).

17. Nelson, C. E. et al. In vivo genome editing improves muscle function in a mouse model of Duchenne muscular dystrophy. Science 351, 403-407, doi:10.1126/science.aad5143 (2016).

18. Long, C. et al. Postnatal genome editing partially restores dystrophin expression in a mouse model of muscular dystrophy. Science 351, 400-403, doi:10.1126/science.aad5725 (2016).

19. Bengtsson, N. E. et al. Muscle-specific CRISPR/Cas9 dystrophin gene editing ameliorates pathophysiology in a mouse model for Duchenne muscular dystrophy. Nature communications 8, 14454, doi:10.1038/ncomms14454 (2017).

20. Maesner, C. C., Almada, A. E. & Wagers, A. J. Established cell surface markers efficiently isolate highly overlapping populations of skeletal muscle satellite cells by fluorescence-activated cell sorting. Skelet Muscle 6, 35, doi:10.1186/s13395-016-0106-6 (2016).

21. Alkass, K. et al. No Evidence for Cardiomyocyte Number Expansion in Preadolescent Mice. Cell 163, 1026-1036, doi:10.1016/j.cell.2015.10.035 (2015).

22. Senyo, S. E. et al. Mammalian heart renewal by pre-existing cardiomyocytes. Nature 493, 433-436, doi:10.1038/nature11682 (2013).

23. Johansen, A. K. et al. Postnatal Cardiac Gene Editing Using CRISPR/Cas9 With AAV9-Mediated Delivery of Short Guide RNAs Results in Mosaic Gene Disruption. Circ Res 121, 1168-1181, doi:10.1161/CIRCRESAHA.116.310370 (2017).

24. Cai, C. L. & Molkentin, J. D. The Elusive Progenitor Cell in Cardiac Regeneration: Slip Slidin' Away. Circ Res 120, 400-406, doi:10.1161/CIRCRESAHA.116.309710 (2017).

25. McGreevy, J. W., Hakim, C. H., McIntosh, M. A. & Duan, D. Animal models of Duchenne muscular dystrophy: from basic mechanisms to gene therapy. Dis Model Mech 8, 195-213, doi:10.1242/dmm.018424 (2015).

26. Wang, L., Wang, H., Bell, P., McMenamin, D. & Wilson, J. M. Hepatic gene transfer in neonatal mice by adeno-associated virus serotype 8 vector. Human gene therapy 23, 533-539, doi:10.1089/hum.2011.183 (2012).

27. Lee, K. et al. Nanoparticle delivery of Cas9 ribonucleoprotein and donor DNA in vivo induces homology-directed DNA repair. Nature Biomedical Engineering 1, 889 (2017).

28. Zinn, E. et al. In Silico Reconstruction of the Viral Evolutionary Lineage Yields a Potent Gene Therapy Vector. Cell Rep 12, 1056-1068, doi:10.1016/j.celrep.2015.07.019 (2015).

1. A method of modifying the genome of a muscle precursor cell in vivo in a subject, comprising contacting the muscle cell with one or more viruses, wherein the one or more viruses
a. transduce a nucleic acid sequence encoding a sequence-targeting nuclease in the muscle precursor cell, and
b. transduce a donor template in the muscle precursor cell,
wherein the modification comprises the insertion of a nucleotide sequence corresponding to a nucleotide sequence of the donor template.

2. The method of claim 1, wherein the one or more viruses comprise a first virus which transduces a nucleic acid sequence encoding a sequence-targeting nuclease and a donor template.

3. The method of claim 1, wherein the one or more viruses comprise a first virus which transduces a nucleic acid sequence encoding a sequence-targeting nuclease, and a second virus which transduces a donor template.

4. The method of claim 1, wherein the one or more viruses comprise a first virus which transduces a nucleic acid sequence encoding a sequence-targeting nuclease, and a second virus which transduces a donor template and one or more gRNAs.

5. The method of claim 1, wherein the sequence-targeting nuclease is a Zinc-Finger Nuclease (ZFN), a Transcription activator-like effector nuclease (TALEN), a Cas nuclease, or a functional fragment or functional variant thereof.

6. (canceled)

7. (canceled)

8. (canceled)

9. The method of claim 1, wherein the muscle precursor cell is a muscle stem cell.

10. (canceled)

11. The method of claim 1, wherein at least 40% of muscle precursor cells in the subject are modified to comprise an insertion of a nucleotide sequence corresponding to a nucleotide sequence of the donor template.

12. The method of claim 1, wherein the virus is AAV serotype 6, 8, 9, 10 or Anc80.

13. The method of claim 1, wherein the subject is a juvenile.

14. (canceled)

15. (canceled)

16. A method of modifying the genome of a cardiac cell in vivo in a subject, comprising contacting the cardiac cell with one or more viruses, wherein the one or more viruses
a. transduce a nucleic acid sequence encoding a sequence-targeting nuclease in the cardiac cell, and
b. transduce a donor template in the cardiac cell,
wherein the modification comprises the insertion of a nucleotide sequence corresponding to a nucleotide sequence of the donor template, and wherein the cardiac cell is a DNA synthesizing cardiac cell or a replicating cardiac cell.

17. The method of claim 16 4-4, wherein the cardiac cell is selected from the group consisting of a mammalian postmitotic cardiomyocyte capable of DNA synthesis without division/proliferation, a human postmitotic cardiomyocyte capable of DNA synthesis without division/proliferation, a cardiomyocyte precursor cell, a proliferating mesenchymal cardiac cell, a proliferating endothelial cardiac cell, and a cardiac progenitor cell.

18. The method of claim 16, wherein the subject is an infant, juvenile, or under 30 years of age.

19. The method of claim 16, wherein the one or more viruses comprise a first virus which transduces a nucleic acid sequence encoding a sequence-targeting nuclease and a donor template.

20. The method of claim 16, wherein the one or more viruses comprise a first virus which transduces a nucleic acid sequence encoding a sequence-targeting nuclease, and a second virus which transduces a donor template.

21. The method of claim 16, wherein the one or more viruses comprise a first virus which transduces a nucleic acid sequence encoding a sequence-targeting nuclease, and a second virus which transduces a donor template and one or more gRNAs.

22. The method of claim 16, wherein the sequence-targeting nuclease is a Zinc-Finger Nuclease (ZFN), a Transcription activator-like effector nuclease (TALEN), a Cas nuclease, or a functional fragment thereof.

23. (canceled)

24. (canceled)

25. The method of claim 16, wherein the virus is AAV serotype 6, 8, 9, 10 or Anc80.

26. The method of claim 16, wherein at least 1.6% of the cardiomyocytes in the subject are modified.

27. (canceled)

28. A method of targeting a specific striated muscle type for genomic modification in vivo in a subject via homology directed repair, comprising systemically administering one or more viruses, wherein the one or more viruses
a. transduce a nucleic acid sequence encoding a sequence-targeting nuclease in striated muscle cells, and
b. transduce a donor template in striated muscle cells,
wherein the modification comprises the insertion of a nucleotide sequence corresponding to a nucleotide sequence of the donor template, and wherein, due to the age of the subject, genomic modification preferentially occurs to at least one type of striated muscle.

29. (canceled)

30. (canceled)

31. The method of claim 28, wherein the subject is an infant, a juvenile, or an adult.
